(19) United States
(12) Patent Application Publication
(10) Pub. No.: US 2010/0260686 A1
(43) Pub. Date: Oct. 14, 2010

(54) Nanoparticles for brain tumor imaging

(75) Inventors: Miqin Zhang, Bothell, WA (US); Conroy Sun, Kirkland, WA (US); Omid Veiseh, Kirkland, WA (US); Narayan Bhattarai, Seattle, WA (US)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(73) Assignee: Washington, University of, Seattle, WA (US)

(21) Appl. No.: 12/384,923

(22) Filed: Apr. 9, 2009

(51) Int. Cl. A61K 49/18; A61K 49/00; A61K 9/16; G01N 33/567

(52) U.S. Cl. 424/9.322; 424/9.1; 424/9.3; 424/493; 435/7.21

(57) ABSTRACT

Nanoparticle having a chitosan-polyethylene oxide oligomer copolymer coating, and methods for making and using the nanoparticle are provided. The nanoparticle can have a core that includes a material that imparts magnetic resonance imaging activity to the particle and, optionally, one or more of an associated targeting agent, fluorescent agent, or therapeutic agent.

### STATEMENT OF GOVERNMENT LICENSE RIGHTS

This invention was made with Government support under Contract Nos. R01CA119408, R01EB006043, and R01CA112350-02 awarded by the National Institutes of Health. The Government has certain rights in the invention.

### BACKGROUND OF THE INVENTION

Nanoparticle-based diagnostic and therapeutic platforms have been investigated extensively due to their potential impact on clinical oncology for improved detection, staging, and treatment of tumors. Among this next generation of molecular imaging agents, magnetofluorescent nanoprobes have attracted significant attention due to their ability to enhance the contrast of tumor regions by both optical and MR imaging. The challenges for in vivo application of nanoprobes for non-invasive tumor imaging include non-specific uptake of nanoprobes by surrounding tissues resulting in poor tumor tissue discrimination. Brain tumor therapy possesses an additional challenge of blood brain barrier (BBB) restriction of circulating nanoprobes.

Magnetic and magnetofluorescent nanoparticles have become important materials for biological applications, especially for sensing, separating, and imaging. Magnetofluorescent nanoparticles have attracted significant attention due to their ability to enhance the contrast of tumor regions in both optical and magnetic resonance imaging. However, in vivo application of nanoparticles for non-invasive tumor imaging has been limited by several challenges, including the nanoparticles' biocompatibility, dispersion, colloidal stability, metabolism, and specificity to the targeted cells.

Many magnetofluorescent nanoparticles suffer from short blood half-life because of their rapid elimination from the blood stream after the injection. Nanoparticles also have a large surface area/volume ratio and therefore tend to agglomerate and adsorb plasma protein. When the nanoparticles agglomerate, or are covered with adsorbed plasma proteins, they are quickly cleared by the action macrophages before they can reach target cells.

Magnetofluorescent nanoparticles are also subject to non-specific uptake by surrounding tissues that results in poor tumor tissue discrimination. For brain tumor patients undergoing surgery, the determination of tumor margins is crucial to successful outcomes. However, the non-specific targeting of many magnetofluorescent nanoparticles prevents surgeons from using these nanoparticles to relate pre-operative radiological images to the visual presentation of pathology during brain tumor surgeries.

Although, nanoparticle-based platforms may substantially improve our ability to non-invasively diagnose, stage, and resect brain tumors, current nanoprobes are limited by insufficient accumulation and retention within tumors due to non-targeting nature, and an inability to traverse the blood brain barrier (BBB). Therefore, a need exists for improved nanoprobes that can traverse the BBB and effectively image brain tumors. The present invention seeks to fulfill this need and provides further related advantages.

### SUMMARY OF THE INVENTION

The present invention provides a nanoparticle having a chitosan-polyethylene oxide oligomer copolymer coating, compositions that include the nanoparticle, and methods for making and using the nanoparticle.

In one aspect of the invention, a nanoparticle is provided. In one embodiment, the nanoparticle comprises:

(a) a core having a surface and comprising a core material; and

(b) a coating on the surface of the core, the coating comprising a copolymer comprising a chitosan and a poly(ethylene oxide) oligomer.

In one embodiment, the core material is a magnetic material. In one embodiment, the copolymer is a graft copolymer having a chitosan backbone and poly(ethylene oxide) oligomer side chains. In other embodiments, the nanoparticle can further include one or more of a targeting agent, a fluorescent agent, and/or a therapeutic agent.

In another aspect, the invention provides a composition, comprising a nanoparticle of the invention and a carrier suitable for administration to a warm-blooded subject.

In a further aspect of the invention, methods for detecting cells or tissues by magnetic resonance imaging are provided. In one embodiment, the method comprises:

(a) contacting cells or tissues of interest with a nanoparticle of the invention having affinity and specificity for the cells or tissues of interest, wherein the nanoparticle comprises
(i) a core comprising a magnetic material and having a surface,
(ii) a coating on the surface of the core, the coating comprising a copolymer comprising a chitosan and a poly(ethylene oxide) oligomer, and
(iii) a targeting agent covalently coupled to the copolymer, wherein the targeting agent has an affinity and specificity to the cells or tissues of interest; and

(b) measuring the level of binding of the nanoparticle, wherein an elevated level of binding, relative to normal cells or tissues, is indicative of binding to the cells or tissues of interest.

In another aspect of the invention, methods for treating a tissue are provided. In one embodiment, the method comprises contacting a tissue of interest with a nanoparticle of the invention having affinity and specificity for the tissue of interest, wherein the nanoparticle comprises
(i) a core comprising a core material and having a surface,
(ii) a coating on the surface of the core, the coating comprising a copolymer comprising a chitosan and a poly(ethylene oxide) oligomer, and
(iii) a targeting agent covalently coupled to the copolymer, wherein the targeting agent has an affinity and specificity to the cells or tissues of interest.

### DESCRIPTION OF THE DRAWINGS

The patent or application file contains at least one drawing executed in color. Copies of this patent or patent application publication with color drawing(s) will be provided by the Office upon request and payment of the necessary fee.

The foregoing aspects and many of the attendant advantages of this invention will become more readily appreciated as the same become better understood by reference to the following detailed description, when taken in conjunction with the accompanying drawings.

FIG. 1 is a schematic illustration of a representative nanoparticle of the invention (NPCP).

FIGS. 2A and 2B present a schematic illustration of the preparation of a representative PEGylated chitosan copolymer (2A) useful in making the nanoparticle of the invention (2B).

FIGS. 3A-3C present a schematic illustration of the synthesis of representative nanoparticles of the invention (NPCP-Cy5.5 and NPCP-Cy5.5-CTX): formation of a reactive chlorotoxin (CTX) conjugate (3A); coupling of Cy5.5 to chitosan-PEG copolymer coated particle (NPCP) to provide NPCP-Cy5.5 (3B); and coupling of the chlorotoxin conjugate to NPCP-Cy5.5 to provide NPCP-Cy5.5-CTX (3C).

FIG. 4 is a bar graph showing the volume-based hydrodynamic size distribution of a representative nanoparticle of the invention (NPCP) measured by dynamic light scattering (DLS).

FIG. 5 is a transmission electron microscopy (TEM) image of a representative nanoparticle of the invention (NPCP) illustrating dispersed iron oxide nanoparticle cores.

FIG. 6 compares the Fourier transform infrared (FTIR) spectra obtained from bare iron oxide nanoparticles (bare NPs), PEG-g-chitosan, and the chitosan-PEG copolymer-coated nanoparticle (NPCP), confirming successful immobilization of PEG-g-chitosan polymer on the iron oxide particle surface.

FIG. 7 is a bar graph showing the effect of chitosan PEGylation on suppression of nanoparticle uptake by microphage cells using iron quantification to evaluate uptake of iron oxide nanoparticles coated with chitosan only (NPC) or with PEG-g-chitosan (NPCP) by RAW 264.7 macrophage cells incubated with 100 μg Fe/ml of NPCP or NPC for 2 hours.

FIGS. 8A and 8B compare in vivo near infrared fluorescence (NIRF) images of autochthonous medulloblastoma tumors in genetically engineered ND2:SmoA1 mice injected with either NPCP-Cy5.5-CTX or NPCP-Cy5.5, or receiving no injection, acquired at 2 hours and 120 hours post injection, respectively. Ex vivo fluorescence images of mice brains from same mice following necropsy are shown in FIG. 8B insets. The scale bar at right corresponds to fluorescence intensity (p/s/cm²/sr) of images.

FIG. 8C is a bar graph comparing the biodistribution of nanoparticles, NPCP-Cy5.5-CTX and NPCP-Cy5.5, in mice obtained through NIRF imaging of stated organs 120 hours post injection.

FIG. 9 is a bar graph comparing the intracellular uptake by 9L glioma cells of representative nanoparticles of the invention (NPCP-CTX) with dextran-coated nanoparticles (NP-Dextran) and non-targeting nanoparticles (NPCP).

FIGS. 10A-10C compare the magnetic properties of the NPCP with the commercial nanoparticle, Feridex IV. FIG. 10A shows the magnetic resonance (MR) images (TR=3000 ms, TE=20 ms) of the NPCP and Feridex IV at concentrations of 0, 2.5, 5, and 10 μg of Fe/ml; FIG. 10B shows the R2 map of the samples depicted in FIG. 10A generated using a series of MR images acquired over a range of echo times (TE) values; and FIG. 10C is a graph illustrating the linear correlation of R2 as a function of Fe concentrations of the NPCP and Feridex IV.

FIGS. 11A and 11B show MR images of coronal cross sections of the frontal lobe of the cerebral hemisphere and cerebellum from symptomatic ND2:SmoA1 mice and wild type mice, respectively, acquired over a range of TEs (14 to 68 ms) prior to and at 48 hours post injection of representative nanoparticles of the invention (NPCP-CTX) or non-targeting NPCP nanoparticles. Colorized R2 maps of the brain region were superimposed onto proton density-weighted images. Varying R2 values (s⁻¹) from low (blue) to high (red) were visually represented in colors generated from the gradient at right.

FIGS. 12A-12D are images of the neurohistopathological examination of brain tissue obtained at 48 hours post-injection from symptomatic ND2:SmoA1 mice (12A and 12B) and wild type mice (12C and 12D) with representative nanoparticles of the invention NPCP-CTX (12A and 12C) and non-targeting NPCP (12B and 12D) each administered through the tail vein.

FIGS. 13A and 13B are bar graphs comparing the MR response quantification determined by dividing the change in R2 before and after the injection of representative nanoparticles of the invention (NPCP-CTX or NPCP) by the pre-injection R2 response for ND2:SmoA1 mice and wild type mice, respectively.

FIGS. 14A and 14B show the images of excised mouse brains and lungs/hearts: from ND2:SmoA1 mice receiving NPCP-Cy5.5-CTX (10 mg/kg, n=3) or no nanoprobe injection (Untreated, n=3), the mice were given Evan's blue dye 3 hours after the nanoparticle injection, sacrificed 2 hours after Evan's blue administration, and perfused with phosphate buffered saline (PBS) (14A); the parasagittal slices of the brain (cerebellum and frontal lobes) from the NPCP-Cy5.5-CTX treated mouse (14B).

FIGS. 15A and 15B show the T1-weighted MR images of brains and kidneys, respectively, of wild type and N2:SmoA1 mice acquired pre-injection and 4 minutes after injection of Gd-DTPA.

FIG. 15C shows T2-weighted MR images of wild type and N2:SmoA1 mice acquired after injection of Gd-DTPA.

FIGS. 15D and 15E show the quantitation of Gd-DTPA accumulations in the tumor, kidney, and healthy tissues (FIG. 15D) and cerebellum and kidney tissues (FIG. 15E) by signal intensities measured from serially acquired T1-weighted MR images of N2:SmoA1 and wild type mice, up to 75 minutes post injection of Gd-DTPA.

FIG. 16 is a graph illustrating a linear correlation between fluorescence intensity and representative nanoparticles of the invention (NPCP-Cy5.5-CTX) concentration (R²=0.9883) using near-infrared fluorescence imaging.

FIG. 17 is a graph comparing in vivo biophotonic fluorescence intensities of medulloblastoma tumors in ND2:SmoA1 mice receiving 200 μg Fe of NPCP-Cy5.5-CTX, NPCP-Cy5.5, or no injection, versus the time period at 2, 24, 48, 96, and 120 hours post nanoparticle injection.

FIG. 18 is a bar graph comparing serum levels of aspartate aminotransferase (AST) and alanine aminotransferase (ALT) in wild type mice injected with NPCP-Cy5.5-CTX, NPCP-Cy5.5, or no injection, measured 120 hours after intravenous administration (mean±standard deviation of the mean, n=3 mice per treatment).

FIGS. 19A and 19B show NPCP-CTX contrast enhanced images of C6/GFP⁺ glioma tumors and corresponding histopathology. FIG. 19A shows MR images (TE range of 46-68 ms) acquired pre- and 48 hours post-administration of NPCP-Cy5.5-CTX (10 mg/kg): proton density weighted MR image (left), pre-injection proton density image with corresponding R2 map overlay (center), and 48 hours post-injection proton density image with corresponding R2 map overlay (right). FIG. 19B shows H&E stained brain section obtained from the same location, in the same plane, as shown in FIG. 19A (scale bar: 1 mm).

FIG. 20 shows the images of H&E stained coronal and axial cross sections from cerebellum of symptomatic ND2:SmoA1 mice confirming presence of medulloblastoma (left) and from cerebellum of wild type mice showing normal cerebellum pathology (right; scale bars: 750 μm).

FIG. 21 shows the images of the Prussian blue/nuclear fast red (iron stain) and H&E stained sections from cerebellum of symptomatic ND2:SmoA1 mice (tumor) and cerebellum of wild type mice (healthy tissue) 5 days post injection of NPCP-Cy5.5-CTX or NPCP-Cy5.5 demonstrating preferential accumulation of NPCP-Cy5.5-CTX in medulloblastoma tumor compared to normal brain tissue and low accumulation of NPCP-Cy5.5 by tumor and normal tissue (scale bars: 50 μm).

FIGS. 22A and 22B show the images of the tissue sections of wild type mice injected with NPCP-Cy5.5-CTX or NPCP-Cy5.5, respectively, and sacrificed at 1, 6, and 24 hour post injection. The tissue sections were stained with a fluorescently labeled antibody against platelet endothelial cell adhesion molecule-1 (PECAM-1; green), a glycoprotein expressed on endothelial cells, and with the nuclear stain DAPI (blue).

FIGS. 23A-23D compare the NIRF and GFP biophotonic fluorescence images of C6/GFP⁺ intracranial glioma tumors acquired 48 hours post injection of representative nanoparticles of the invention. FIG. 23A compares in vivo NIRF optical images of control (S) and C6/GFP⁺ tumor-bearing (T) mice receiving NPCP-Cy5.5-CTX, NPCP-Cy5.5, or no injection; FIGS. 23B and 23C compare ex vivo NIRF (first row) and GFP (second row) images of whole intact brains removed from these mice and brains sectioned along the sagittal plane from these mouse brains, respectively; FIG. 23D compares the confocal fluorescent images of the brain sections from the C6/GFP⁺ mice injected with NPCP-Cy5.5-CTX and nuclei-stained with DAPI (scale bar: 20 μm).

### DETAILED DESCRIPTION OF THE INVENTION

The present invention provides a nanoparticle having a chitosan-polyethylene oxide oligomer copolymer coating. In certain embodiments, the nanoparticle has a core that includes a material that imparts magnetic resonance imaging activity to the particle. The nanoparticle can further include one or more of a targeting agent to target the nanoparticle to a site of interest, a fluorescent agent that allows for fluorescence imaging of the particle, or a therapeutic agent that can be delivered by the particle. The targeting, fluorescent, and therapeutic agents can be coupled to the particle's copolymer coating. Methods for making and using the nanoparticles are also provided.

In one aspect, the invention provides a nanoparticle. The nanoparticle has a core comprising a core material, and a copolymer coating on the core surface. The coating comprises a copolymer comprising a chitosan and a poly(ethylene oxide) oligomer.

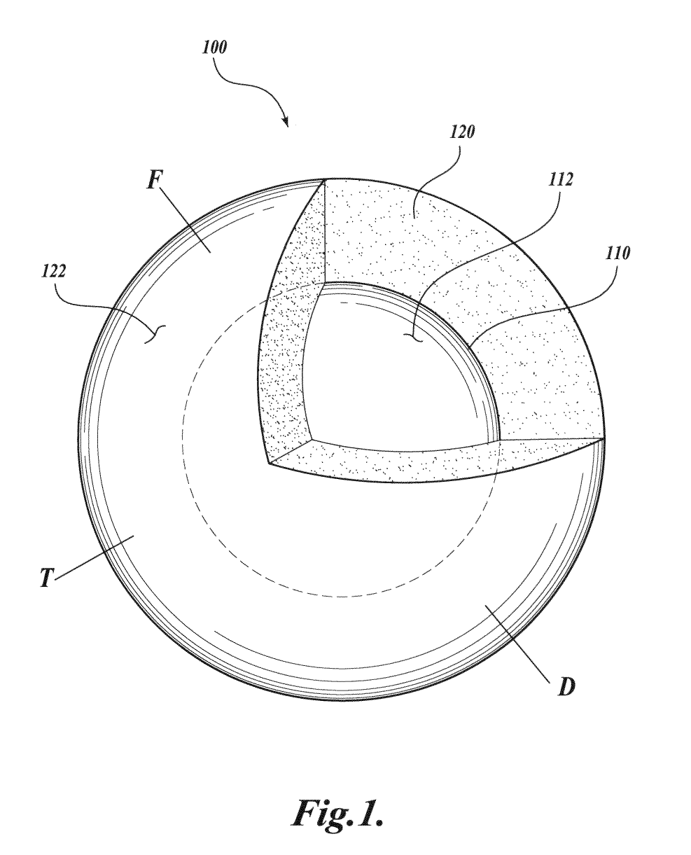

A schematic illustration of a representative nanoparticle of the invention is shown in FIG. 1. Referring to FIG. 1, particle 100 includes core 110 having surface 112 with coating 120 encapsulating the core.

The copolymer forms a coating on the core surface. The copolymer is anchored to the core surface (e.g., oxide surface) by interactions between the core surface and the amine and hydroxyl groups on the copolymer's chitosan backbone. It is believed that the coating is a multi-layered mesh that encapsulates the core.

The coating of the nanoparticle is formed from a copolymer comprising a chitosan and a poly(ethylene oxide) oligomer. In one embodiment, the copolymer is a graft copolymer having a chitosan backbone and pendant poly(ethylene oxide) oligomer side chains.

Chitosan is a linear polysaccharide composed of randomly distributed β-(1-4)-linked D-glucosamine (deacetylated unit) and N-acetyl-D-glucosamine (acetylated unit). Suitable chitosans useful in making the copolymers useful in the invention have a molecular weight (weight average, Mw) of from about 0.3 to about 50 kDa. In certain embodiments, the chitosan has a molecular weight of from about 0.5 to about 15 kDa. In one embodiment, the chitosan has a molecular weight of about 10 kDa. Suitable chitosans include oxidatively degraded chitosans prepared from commercially available chitosan as described in Example 1.

The copolymer also includes a plurality of poly(ethylene oxide) oligomers. In one embodiment, poly(ethylene oxide) oligomers are grafted to the chitosan's backbone to provide a copolymer having pendant poly(ethylene oxide) oligomer side chains.

Suitable poly(ethylene oxide) oligomers include poly(ethylene oxides) (PEO or PEG) and poly(ethylene oxide) copolymers such as block copolymers that include poly(ethylene oxide) and poly(propylene oxide) (e.g., PEO-PPO and PEO-PPO-PEO). In one embodiment, the poly(ethylene oxide) oligomer is a poly(ethylene oxide). In certain embodiments, poly(ethylene oxide) oligomer has a molecular weight (weight average, Mw) of from about 0.3 to about 40 kDa. In others embodiments, the poly(ethylene oxide) oligomer has a molecular weight of from about 1.0 to about 10 kDa. In certain embodiments, the poly(ethylene oxide) oligomer has a molecular weight of about 2 kDa.

Representative chitosan-poly(ethylene oxide) oligomer copolymers include from about 2 to about 50 weight percent poly(ethylene oxide) oligomer. In one embodiment, the copolymer includes from about 5 to about 25 weight percent poly(ethylene oxide) oligomer.

Representative chitosan-poly(ethylene oxide) oligomer graft copolymers have a degree of poly(ethylene oxide) oligomer substitution of from about 0.01 to about 0.5. In certain embodiments, the graft copolymers have a degree of poly(ethylene oxide) oligomer substitution from about 0.01 to about 0.2. As used herein, the term “degree of substitution” or “DS” refers to the fraction of glucosamine repeating units in the chitosan that are substituted with a poly(ethylene oxide) oligomer. For DS=1.0, 100% of the glucosamine units are substituted with the poly(ethylene oxide) oligomer.

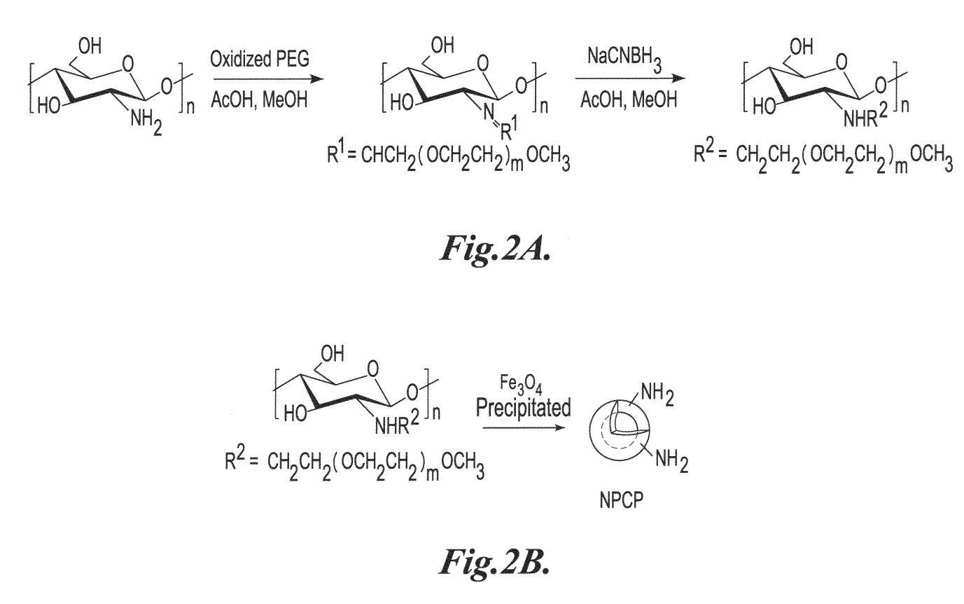

The preparation of representative copolymers useful in the nanoparticle of the invention is described in Example 1 and schematically illustrated in FIGS. 2A and 2B. FIG. 2A depicts reaction of the amino group (—NH₂) of the glucosamine repeating unit of a suitable chitosan with a suitably reactive poly(ethylene oxide) oligomer (PEG-CHO, oxidized PEG) to provide a Schiff base adduct (═N—R¹) that is than reduced to provide the PEG grafted chitosan (—NHR²). It will be appreciated that FIGS. 2A and 2B depict the chitosan having the poly(ethylene oxide) oligomer grafted to its repeating unit, the degree of substitution for the chitosan is as described above.

The nanoparticle includes a core material. For magnetic resonance imaging applications, the core material is a material having magnetic resonance imaging activity (e.g., the material is paramagnetic). In certain embodiments, the core material is a magnetic material. In other embodiments, the core material is a semiconductor material. Representative core materials include ferrous oxide, ferric oxide, silicon oxide, polycrystalline silicon oxide, silicon nitride, aluminum oxide, germanium oxide, zinc selenide, tin dioxide, titanium, titanium dioxide, nickel titanium, indium tin oxide, gadolinium oxide, stainless steel, gold, and mixtures thereof.

As noted above, the nanoparticle is prepared by coating the core with the copolymer. The preparation of a representative nanoparticle of the invention (NPCP) is described in Example 1 and illustrated schematically in FIG. 2B. In a representative method, the nanoparticle is formed by co-precipitation of iron oxide and the copolymer.

The particle of the invention has nanoscale dimensions. Suitable particles have a physical size less than about 50 nm. In certain embodiments, the nanoparticles have a physical size from about 2 to about 30 nm. As used herein, the term “physical size” refers the overall diameter of the nanoparticle, including core (as determined by TEM) and coating thickness. Suitable particles have a mean core size of from about 2 to about 25 nm. In certain embodiments, the nanoparticles have a mean core size of about 7 nm. As used herein, the term “mean core size” refers to the core size determined by TEM. Suitable particles have a hydrodynamic size less than about 250 nm. In certain embodiments, the nanoparticles have a hydrodynamic size from about 10 to about 250 nm. In certain embodiments, the nanoparticles have a hydrodynamic size of about 33 nm. As used herein, the term “hydrodynamic size” refers the radius of a hard sphere that diffuses at the same rate as the particle under examination as measured by DLS. The hydrodynamic radius is calculated using the particle diffusion coefficient and the Stokes-Einstein equation given below, where k is the Boltzmann constant, T is the temperature, and η is the dispersant viscosity:

$${R}_{H} = \frac{kT}{6 π η D} .$$

A single exponential or Cumulant fit of the correlation curve is the fitting procedure recommended by the International Standards Organization (ISO). The hydrodynamic size extracted using this method is an intensity weighted average called the Z average.

The nanoparticles of the invention include the copolymer coated nanoparticles described above that further include one or more other agents. Thus, in other embodiments, the nanoparticles of the invention further include one or more of a targeting agent to target the nanoparticle to a site of interest, a fluorescent agent that allows for fluorescence imaging of the particle, or a therapeutic agent that can be delivered by the particle. The targeting, fluorescent, and therapeutic agents can be coupled to the particle's copolymer coating. FIG. 1 schematically illustrates a nanoparticle of the invention that includes a targeting agent (T), a fluorescent agent (F), and a therapeutic agent (D).

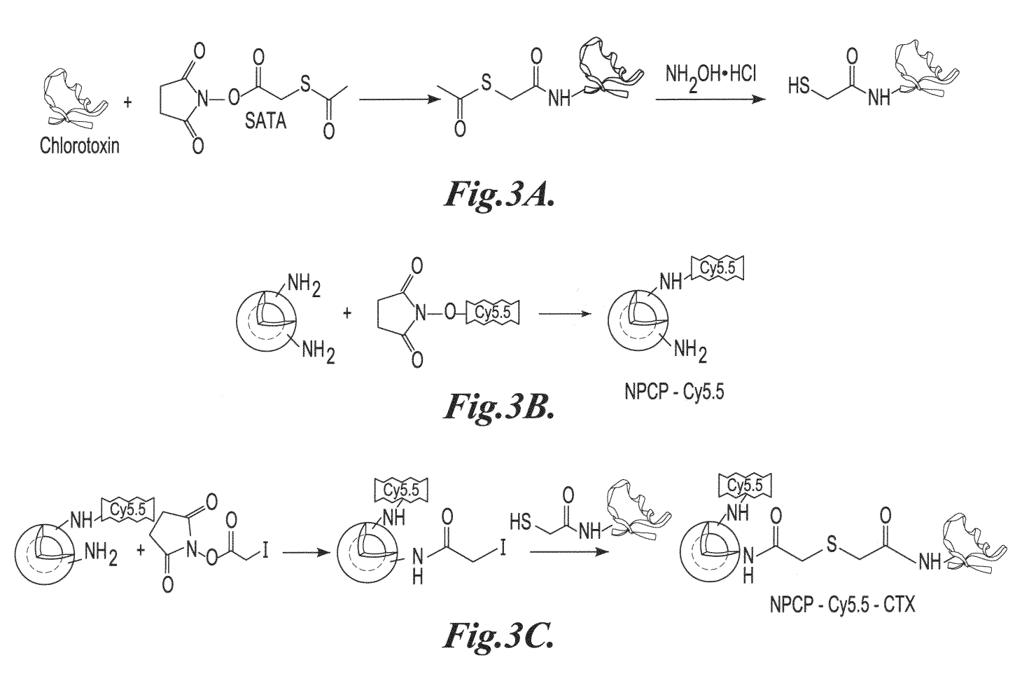

The preparation and characteristics of representative nanoparticles of the invention that include fluorescent and targeting agents are described in Example 1 and illustrated schematically in FIGS. 3A-3C. The cellular uptake of representative nanoparticles in vitro is described in Example 2. The evaluation of representative nanoparticles in vivo is described in Example 3.

Suitable targeting agents include compounds and molecules that direct the nanoparticle to the site of interest. Representative targeting agents include small molecules, peptides, proteins, and nucleic acids (e.g., DNA, RNA, cDNA, siRNA). Representative small molecule targeting agents include vitamins and hormones. In one embodiment, the targeting agent is chlorotoxin.

In one embodiment, the invention provides a nanoparticle, comprising:

(a) a core comprising a magnetic material and having a surface;

(b) a coating on the surface of the core, the coating comprising a copolymer comprising a chitosan and a poly(ethylene oxide) oligomer; and

(c) a targeting agent covalently coupled to the copolymer.

Suitable fluorescent agents include fluorescent agents that emit light in the visible and near-infrared (e.g., fluorescein and cyanine derivatives). Representative fluorescent agents include fluorescein, OREGON GREEN 488, ALEXA FLUOR 555, ALEXA FLUOR 647, ALEXA FLUOR 680, Cy5, Cy5.5, and Cy7.

In one embodiment, the invention provides a nanoparticle, comprising:

(a) a core comprising a magnetic material and having a surface; and

(b) a coating on the surface of the core, the coating comprising a copolymer comprising a chitosan and a poly(ethylene oxide) oligomer;

(c) a targeting agent covalently coupled to the copolymer; and

(d) a fluorescent agent covalently coupled to the copolymer.

Suitable therapeutic agents include conventional therapeutic agents, such as small molecules; biotherapeutic agents, such as peptides, proteins, and nucleic acids (e.g., DNA, RNA, cDNA, siRNA); and cytotoxic agents, such as alkylating agents, purine antagonists, pyrimidine antagonists, plant alkaloids, intercalating antibiotics, aromatase inhibitors, anti-metabolites, mitotic inhibitors, growth factor inhibitors, cell cycle inhibitors, enzymes, topoisomerase inhibitors, biological response modifiers, anti-hormones, and anti-androgens. Representative cytotoxic agents include BCNU, cisplatin, gemcitabine, hydroxyurea, paclitaxel, temozomide, topotecan, fluorouracil, vincristine, vinblastine, procarbazine, dacarbazine, altretamine, cisplatin, methotrexate, mercaptopurine, thioguanine, fludarabine phosphate, cladribine, pentostatin, fluorouracil, cytarabine, azacitidine, vinblastine, vincristine, etoposide, teniposide, irinotecan, docetaxel, doxorubicin, daunorubicin, dactinomycin, idarubicin, plicamycin, mitomycin, bleomycin, tamoxifen, flutamide, leuprolide, goserelin, aminoglutethimide, anastrozole, amsacrine, asparaginase, mitoxantrone, mitotane, and amifostine.

In one embodiment, the invention provides a nanoparticle, comprising:

(a) a core comprising a magnetic material and having a surface; and

(b) a coating on the surface of the core, the coating comprising a copolymer comprising a chitosan and a poly(ethylene oxide) oligomer;

(c) a targeting agent covalently coupled to the copolymer; and

(d) a therapeutic agent.

In the above embodiment, the therapeutic agent can be covalently coupled to the copolymer or non-covalently (e.g., ionic) associated with the copolymer. For therapeutic agent delivery, the therapeutic agent can be covalently linked (e.g., through a cleavable linkage), physically adsorbed to (e.g., electrostatic or van der Waals interactions), or embedded within the nanoparticle's copolymer coating.

In another aspect of the invention, a composition is provided that includes a nanoparticle of the invention and a carrier suitable for administration to a human subject. Suitable carriers include those suitable for intravenous inject (e.g., saline or dextrose).

In other aspects, the invention provides methods for using the nanoparticles of the invention. The methods include imaging methods such as magnetic resonance imaging when the core has magnetic resonance activity, and optical imaging when the nanoparticle includes a fluorescent agent. The nanoparticles of the invention can also be used for drug delivery when the nanoparticle includes a therapeutic agent. For nanoparticles of the invention that include targeting agents, imaging of and drug delivery to target sites of interest are provided.

In one embodiment, the invention provides a method for detecting (or imaging) cells or tissues by magnetic resonance imaging, comprising:

(a) contacting cells or tissues of interest with a nanoparticle of the invention having affinity and specificity for the cells or tissues of interest, wherein the nanoparticle comprises
(i) a core comprising a magnetic material and having a surface,
(ii) a coating on the surface of the core, the coating comprising a copolymer comprising a chitosan and a poly(ethylene oxide) oligomer, and
(iii) a targeting agent covalently coupled to the copolymer, wherein the targeting agent has an affinity and specificity to the cells or tissues of interest; and

(b) measuring the level of binding of the nanoparticle to the cells or tissues of interest, wherein an elevated level of binding, relative to normal cells or tissues, is indicative of binding to the cells or tissues of interest.

In the method, the level of binding is measured by magnetic resonance imaging techniques. In a further embodiment of the above method, the nanoparticle further includes a fluorescent agent. In this embodiment, the level of binding can be measured by magnetic resonance and/or fluorescence imaging techniques. The methods are applicable to detecting or imaging cells or tissues in vitro. The methods are also applicable to detecting or imaging cells or tissues in vivo. In this embodiment, the nanoparticles are administered to a subject (e.g., warm-blooded animal) by, for example, intravenous injection.

In another embodiment, the invention provides a method for treating a tissue, comprising contacting a tissue of interest with a nanoparticle of the invention having affinity and specificity for the tissue of interest, wherein the nanoparticle comprises
(i) a core comprising a core material and having a surface,
(ii) a coating on the surface of the core, the coating comprising a copolymer comprising a chitosan and a poly(ethylene oxide) oligomer, and
(iii) a targeting agent covalently coupled to the copolymer, wherein the targeting agent has an affinity and specificity to the cells or tissues of interest.

In a further embodiment of the above method, the nanoparticle further comprises a therapeutic agent. In this embodiment, the therapeutic agent can be covalently linked (e.g., through a cleavable linkage), physically adsorbed to (e.g., electrostatic or van der Waals interactions), or embedded within the nanoparticle's copolymer coating. The methods are applicable to treating tissues in vitro. The methods are also applicable to treating tissues in vivo. In this embodiment, the nanoparticles are administered to a subject (e.g., warm-blooded animal) by, for example, intravenous injection.

The following is a description of specific nanoparticles of the invention and methods for their use.

Biocompatibility, dispersion, colloidal stability, and targeted delivery have long been technological challenges in the development of effective nanoparticle-based diagnostic and therapeutic technologies. Moreover, the delivery of nanoparticles to tumors within the central nervous system poses even stricter requirements on their physical and chemistry properties to ensure their passage across the BBB. The present invention provides a nanoprobe having a demonstrated mobility to across the BBB to target brain tumors in vivo, and has both MR and optical imaging functionality.

To improve the performance of these imaging contrast agents (i.e. reduce non-specific uptake and increase permeation through biological barriers), extensive effort was dedicated toward minimizing the overall size of the nanoprobe and enhancing its chemical and biological stability. Design elements, such as synthesizing core nanoparticles with minimal size, coating nanoparticles with a thin yet dense polymer layer, integrating ample chemical functionality, employing a small tumor-specific ligand, and use of biocompatible materials, were all integrated in the development scheme of the nanoprobe of the invention. The combination of these components provided a versatile nanoprobe platform with the potential for expansion into a wide range of applications in clinical oncology, such as targeted drug delivery and detection of various tumor types.

The physiochemical properties, such as hydrodynamic size, charge, and surface chemistry, dictate the pharmacokinetics and biodistribution of nanoparticles in vivo. Due to their high surface energy, as a result of a large surface area to volume ratio, bare nanoparticles tend to agglomerate and adsorb plasma proteins. These aggregates and opsonized nanoparticles are readily recognized by the immune system and are rapidly cleared from the bloodstream prior to reaching target tissues. Thus, inorganic nanoparticles, such as iron oxide nanocrystals, are typically coated with polymers to increase particle dispersion and suppress opsonization. In the nanoparticles of the invention, PEG-grafted chitosan serves as a polymeric coating functioning both as a linker and stabilizer. The inherent natural functionality of chitosan's amine and hydroxyl groups provide convenient handles for conjugation of multiple and varying types of ligands.

In one aspect, the invention provides a nanoprobe in which the base nanoparticle is comprised of an iron oxide nanoparticle coated with a PEGylated-chitosan branched copolymer (see FIG. 1), herein to referred to as “NPCP.” In embodiment, chitosan is utilized as a linker and stabilizer. The amino and hydroxyl groups of chitosan's glucosamine backbone serve to anchor the polymer to the iron oxide surface alleviating the need for crosslinking agents while providing sites for conjugation of ligands (e.g., fluorescent agents F, targeting agents T, and therapeutic drugs D) without the need for further chemical modification. The bound chitosan also acts as a sterically stabilizing corona, preventing particle aggregation under physiological conditions. PEG was integrated into the polymer coating to reduce protein adsorption, limit immune recognition, and thereby increase the nanoprobe blood half-life in vivo. The NPCP received selective targeting and optical functionality via covalently linked chlorotoxin (CTX) and the near-infrared fluorophore, Cy5.5, respectively. In vivo targeting efficacy of these NPCP-based contrast agents was evaluated using the transgenic mouse model, ND2:SmoA1, that closely resembles human medulloblastoma, the most common malignant childhood brain tumor. While maintaining an intact BBB, medulloblastomas arise spontaneously in the cerebellum due to transgenic expression of constitutively active smoothened, a mediator of sonic hedgehog activity. The developed nanoprobe was also tested in an intracranial C6 glioma mouse model to evaluate nanoprobe targeting specificity against an alternative tumor model. Through in vivo MR and optical imaging, and histological study, the ability of these nanoprobes to cross the BBB, and specifically target medulloblastomas and gliomas, was demonstrated.

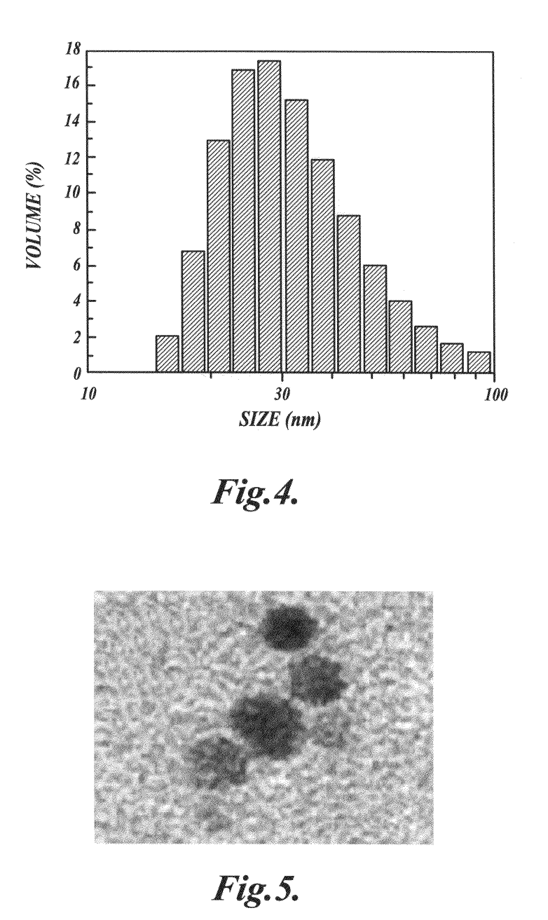

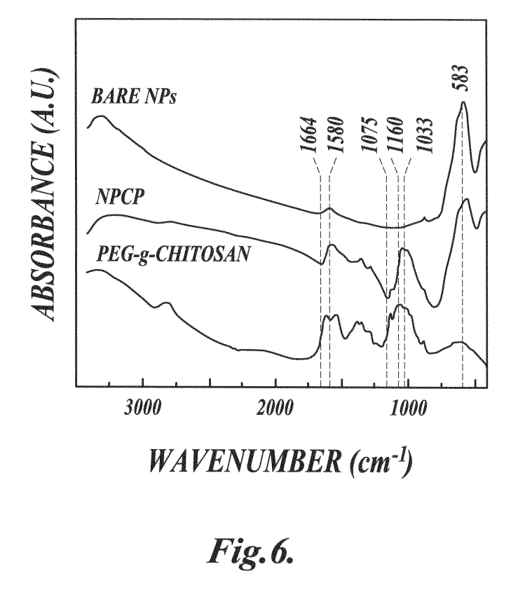

The base NPCP was synthesized through a co-precipitation process and simultaneously coated in situ with a PEG-grafted chitosan copolymer synthesized by alkylation of chitosan followed by Schiff base formation and reduction (see FIG. 2A). During NPCP synthesis (see FIG. 2B), the polymer serves to limit crystal growth, thereby controlling particle size and morphology. The mean hydrodynamic size of the resulting NPCPs was found to be 33 nm by dynamic light scattering (DLS) (FIG. 4). Transmission electron microscopy (TEM) of the NPCP showed the dispersed iron oxide cores with a relatively uniform shape and a mean diameter of 7 nm (FIG. 5). Powder X-ray diffraction (XRD) of the NPCP was consistent with that of crystalline magnetite (Fe₃O₄; JCPDS card No. 19-0629). The successful immobilization of the copolymer on the iron oxide surface was confirmed by Fourier transform infrared spectroscopy (FTIR) (FIG. 6). CTX and Cy5.5 were then immobilized on the NPCP surface via amino groups of the copolymer coating to provide 16.2 CTX peptides and 1.5 fluorophores per NPCP (see FIGS. 3A-3B. The zeta potential of the produced tumor-targeting nanoprobe was 4.2 mV.

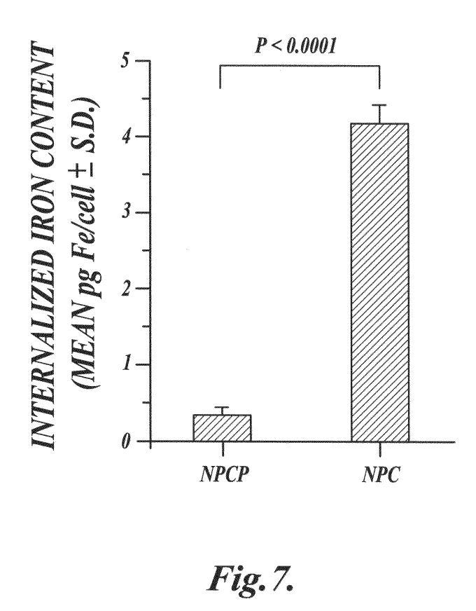

In vivo application of nanoparticles coated with chitosan (not PEG-grafted) is limited by a high positive charge resulting in plasma protein adsorption, followed by clearance from the bloodstream by the reticuloendothelial system (RES) before reaching target tissues. To reduce nanoprobe isolation by macrophage cells of the RES, PEG was integrated into the nanoprobe coating. A 12.4±0.7 (mean±s.d. P<0.0001) fold decrease in nanoparticle uptake was observed, in vitro, by RAW 264.7 macrophages incubated with NPCP compared with iron oxide nanoparticles coated with chitosan alone (NPC) (FIG. 7), improving the nanoprobe's potential for in vivo use.

Commonly found in FDA-approved formulations, chitosan and its derivatives are used in a wide range of applications throughout the biotechnology, medical, cosmetics, and food industries. However, chitosan's applications are limited due to its limited solubility in neutral aqueous solutions, its tendency to agglomerate in high ionic strength solvents, and its limited blood half-life as a result of its cationic profile. To circumvent these limitations, PEG was grafted onto chitosan to increase its hydrophilicity and reduce its positive charge. Well established in the literature for its anti-fouling properties, PEG exhibits steric repulsion that can reduce protein adsorption, preventing opsonization and phagocytosis of the nanoprobes in vivo, resulting in an increased blood half-life. In addition, the amphiphilic nature of PEG molecules was integrated into the nanoprobe design to assist in crossing the BBB by facilitating their transcytosis through endothelial cells that comprise brain capillaries. As noted above, through incorporation of PEG into the nanoprobe's polymer coating, the biocompatibility of the nanoprobe was improved and uptake of the nanoprobe by macrophage cells was substantially reduced (FIG. 7). Furthermore, the addition of PEG to the nanoprobe coating greatly increased the colloidal stability of the nanoprobe. Nanoparticles coated with PEG-grafted chitosan were found to be stable (i.e., no agglomeration or loss of functionality) in solutions of physiological pH for months compared to those coated with chitosan alone, which demonstrated a short shelf-life of a few hours under identical conditions.

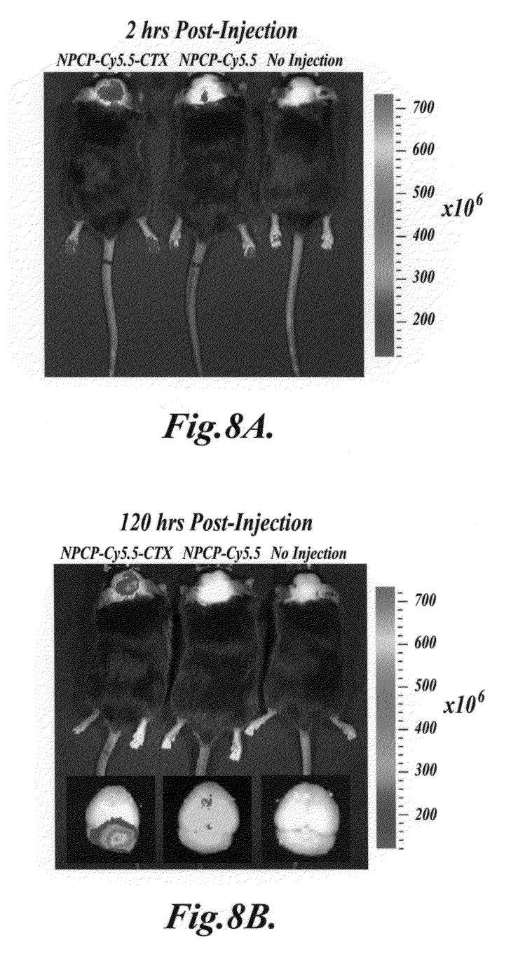

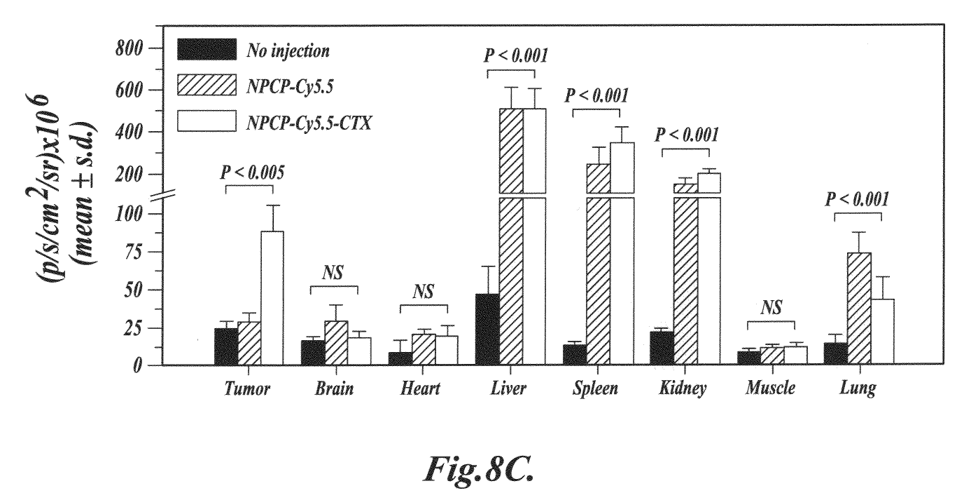

The significance of specific targeting in tumor contrast enhancement was demonstrated by optical imaging where non-targeting nanoparticles showed no specificity for intracranial tissues in tumor-bearing mice (FIG. 8C). Cell targeting ensures that nanoprobes are preferentially taken up by tumor cells rather than normal tissue, consequently improving target-to-background ratios. This specificity allows for low dosage requirements to achieve adequate contrast enhancement, minimizing negative side effects of the nanoprobe against normal tissue. This is particularly crucial for drug delivery applications, where nanoparticles carry harmful therapeutic payloads. In one embodiment, chlorotoxin (CTX) was utilized as a targeting agent due to its specific MMP-2 facilitated binding, a target highly expressed on primary tumors of neuroectodermal origin. A significant difference in nanoprobe accumulation in tumors over normal tissue and between targeted (NPCP-Cy5.5-CTX) and non-targeted (NPCP-Cy5.5) delivery (FIGS. 8A and 8B) was demonstrated. Unlike antibodies or alternative targeting ligands, which are limited to individual types of brain tumors, the molecular target for CTX is expressed in a majority of brain tumors as well as a variety of other tumors. Use of the CTX peptide as a targeting ligand also minimizes the overall size of the nanoprobe due to its small size.

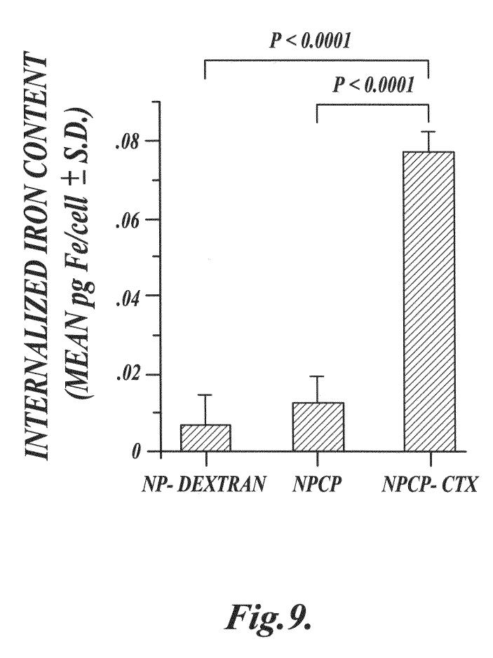

In one embodiment, CTX was employed as a targeting agent due to its specific binding to primary tumors of the neuroectordermal origin via MMP-2 dependent binding. The targeting specificity of NPCP conjugated with CTX (NPCP-CTX) was confirmed in vitro by preferential uptake of the nanoprobe by 9L rat gliosarcoma cells, which demonstrated a 6.1±1.1 (mean±s.d. P<0.0001) fold increase in uptake compared with non-targeting control nanoprobes, and an 11±0.8 (mean±s.d. P<0.0001) fold increase compared with dextran-coated nanoparticles (FIG. 9).

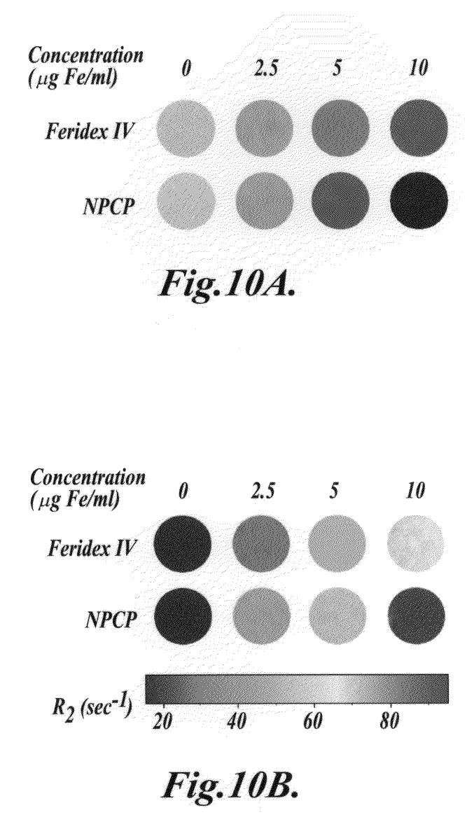

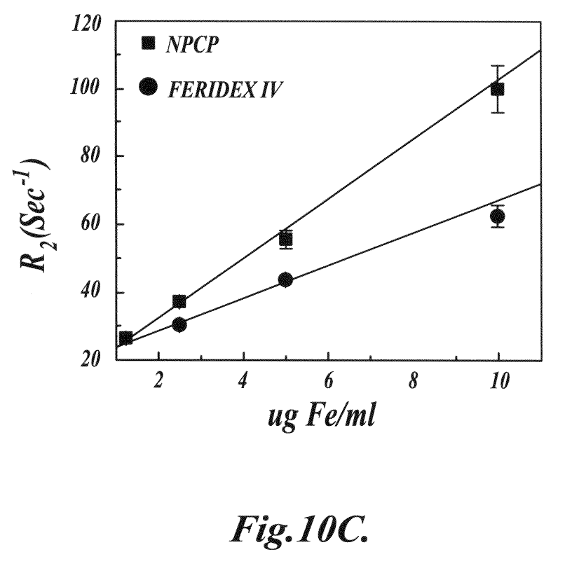

Spin-spin relaxation times (T2) were obtained from phantom images of agarose samples containing NPCP of varying concentrations (FIG. 10A). The magnetic properties of NPCP were evaluated with the commercially available Feridex IV (a dextran-coated nanoparticle) as a reference. Utilizing MR images acquired over a range of echo times (TE) to generate a R2 (1/T2) map (FIG. 10B), a linear correlation of R2 with iron concentration was established for both contrast agents (FIG. 10C). The R2 relaxivities were 472.3 s⁻¹ mM⁻¹ and 243.3 s⁻¹ mM⁻¹ for NPCP and Feridex IV, respectively. The substantially higher relaxation effect exhibited by NPCP over Feridex IV is believed to be due to differences in core iron oxide crystal size as well as the thickness and nature of the coating material used in the two systems. These factors affect the relaxation of water protons closely surrounding the nanoparticle resulting in differing levels of contrast enhancement.

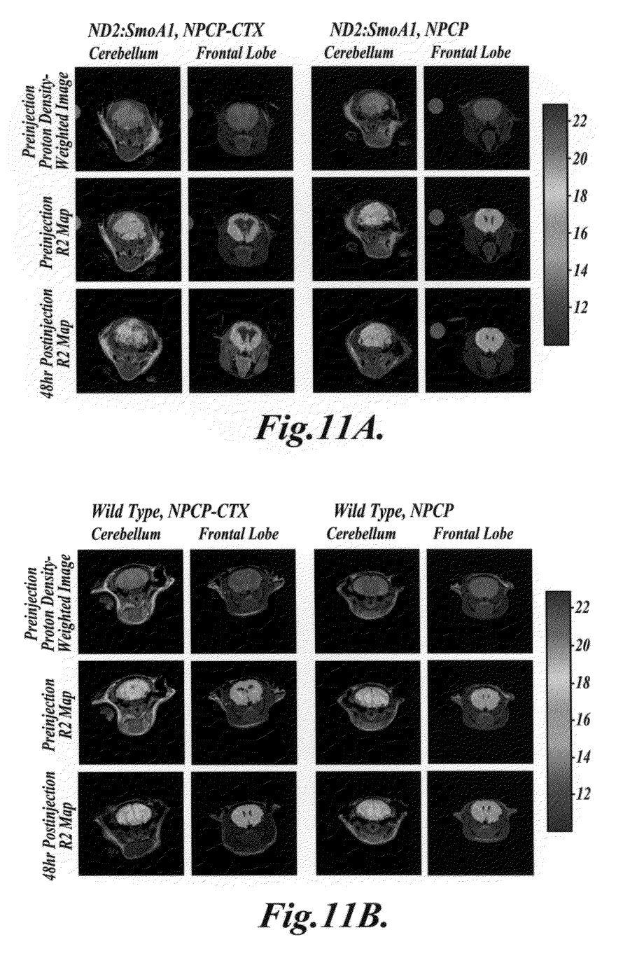

In vivo MR imaging experiments were performed on both symptomatic ND2:SmoA1 mice and wild type mice with NPCP-CTX or non-targeting NPCP nanoprobe administrations, each administered through the tail vein. MR images were acquired over a range of TEs (14 to 68 ms) prior to injection and at 48 hrs post-injection (FIGS. 11A and 11B). Coronal scans of the frontal lobe of the cerebral hemisphere (healthy tissue) and cerebellum (tumor-containing tissue) were analyzed for nanoparticle localization. Proton density-weighted images prior to nanoprobe administration, and R2 maps prior to and post nanoprobe administration were acquired. Increased R2 relaxivities of 48 hr time point scans over preinjection images is indicative of regions of nanoprobe accumulation.

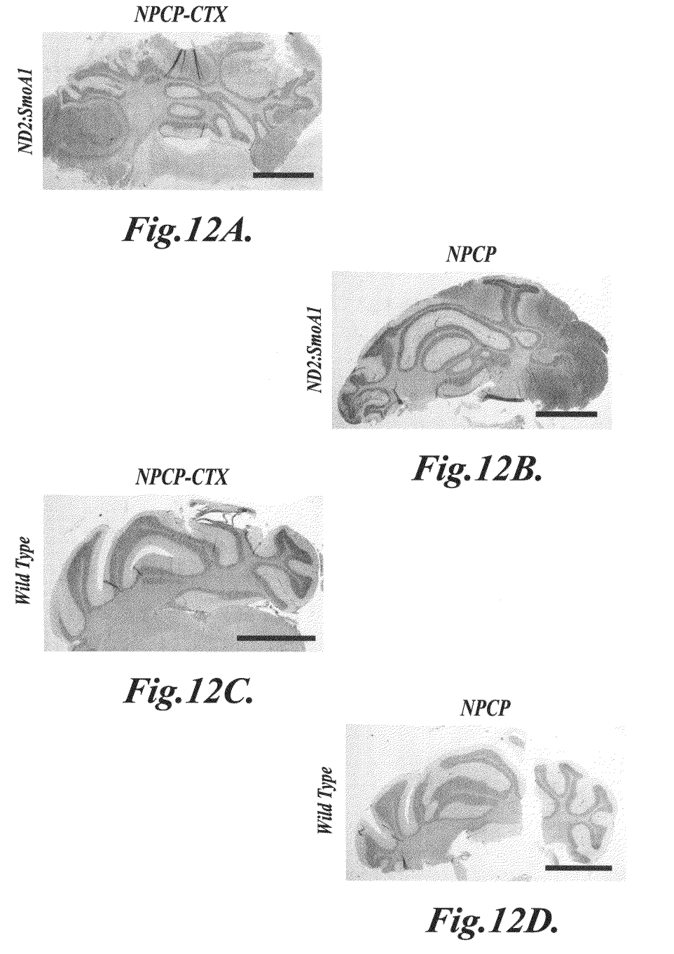

R2 maps generated from proton density-weighted images of ND2:SmoA1 mice indicated slight T2 variability of the cerebellum prior to nanoprobe administration. 48 hrs post NPCP-CTX injection, significantly increased contrast (red coloration) at the periphery of the cerebellum indicated specific nanoparticle localization. These highlighted regions compare favorably with the tumor regions identified in histological sections of the same slices stained with haematoxylin and eosin (H&E) (FIGS. 12A-12D). Slight variations between R2 maps and histology samples can be attributed to differences in sample slice depths and locations of MR imaging and histological sectioning. Similarities were not observed in ND2:SmoA1 mice treated with non-targeting NPCP. In addition, the frontal lobe region did not show significant R2 shifts between pre-injection and post-injection imaging, indicating limited or no nanoprobe accumulation in healthy brain.

Preferential targeting of the NPCP-CTX nanoprobe was further illustrated as wild type mice injected with NPCP-CTX nanoprobes showed negligible accumulation in the brain (FIG. 11B), and the non-targeting control nanoparticle, NPCP, showed no apparent localization (i.e., change in R2 relaxivity) in either the cerebellum or frontal lobe of ND2:SmoA1 and wild type mice (FIGS. 11A and 11B).

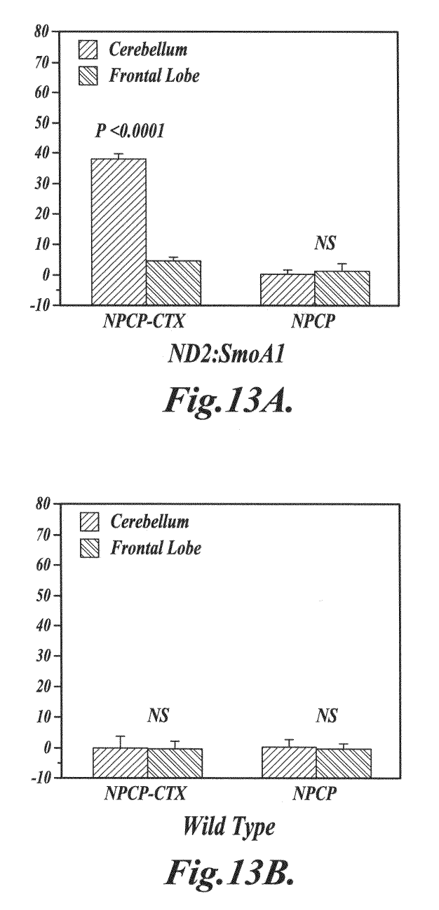

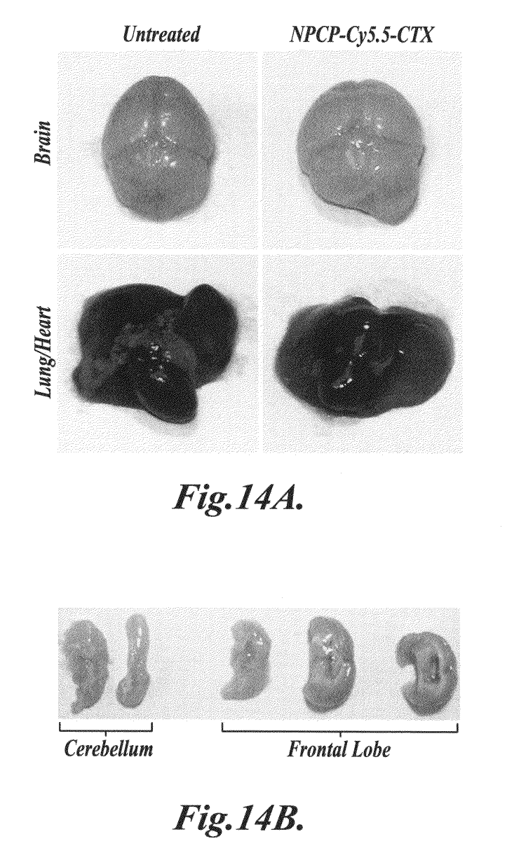

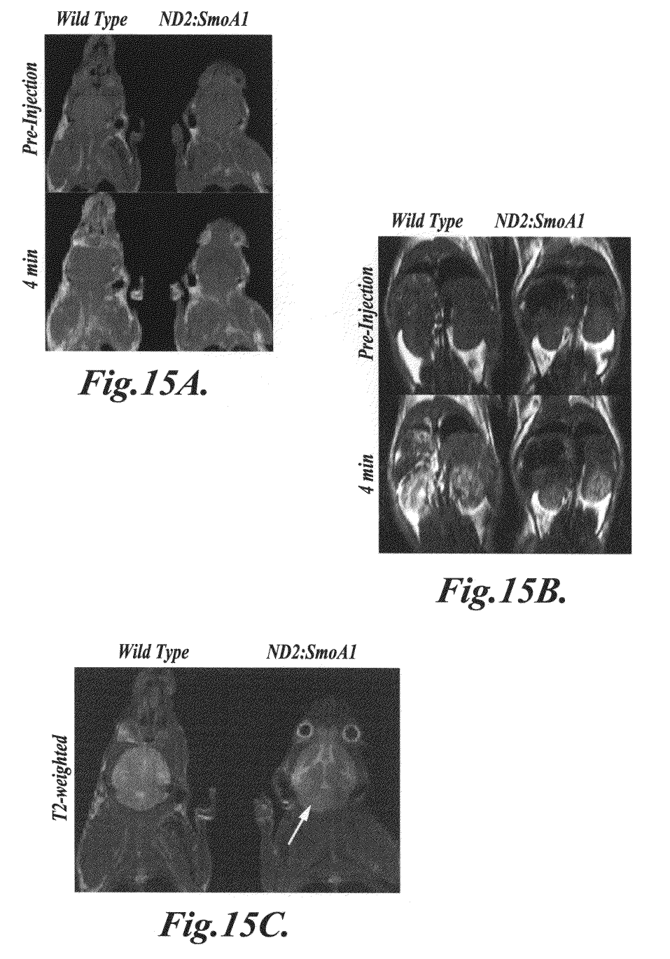

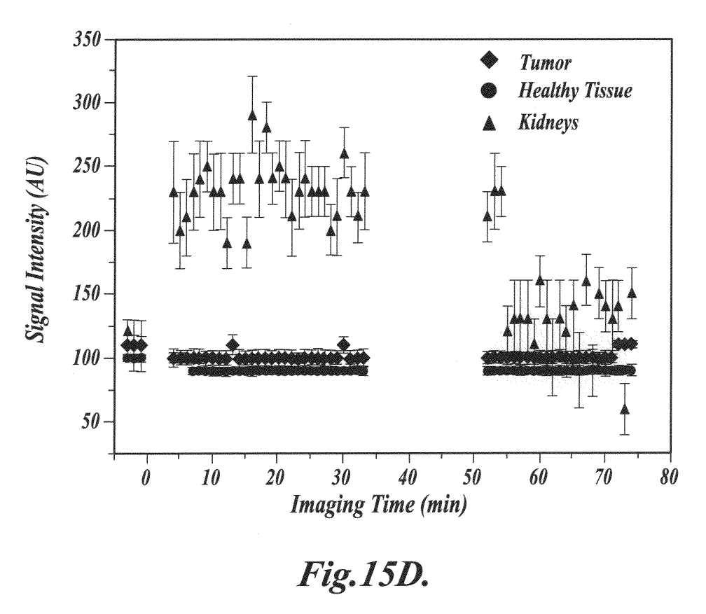

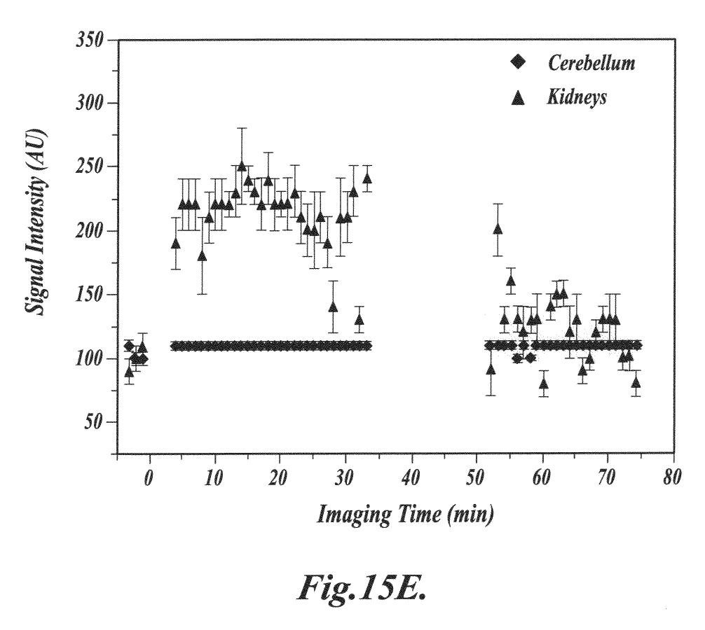

MR response quantification, determined by dividing the change in R2 before and after nanoprobe injection by the pre-injection R2 response (FIGS. 13A and 13B), confirmed the qualitative analysis. Upon administration of the targeting NPCP-CTX probe, contrast enhancement of 37.6±2.4% (mean±s.d.) was observed in the cerebellum (FIG. 13A), significantly higher than that of the healthy tissue of the frontal lobe (4.3±1.6%, P<0.0001). In comparison, post-injection contrast enhancement of the cerebellum of non-targeting NPCP-treated mice was 0.7±0.9%, a minimal R2 change similar to that of the frontal lobe (1.2±2.5%, P>0.05). Significantly, no major change in R2 was observed in either frontal lobe (P>0.05) or cerebellum (P>0.05) regions of wild type mice injected with either nanoprobe (FIG. 13B). Integrity of the BBB of ND2:SmoA1 mice was confirmed by exclusion assays of Evan's blue and the Gadolinium-DTPA complex (FIGS. 14A-14B, and 15A-15E, respectively). These results suggest that the nanoprobe is capable of reaching and accumulating in medulloblastoma tissue across the intact BBB, providing specific contrast enhancement of tumor tissue.

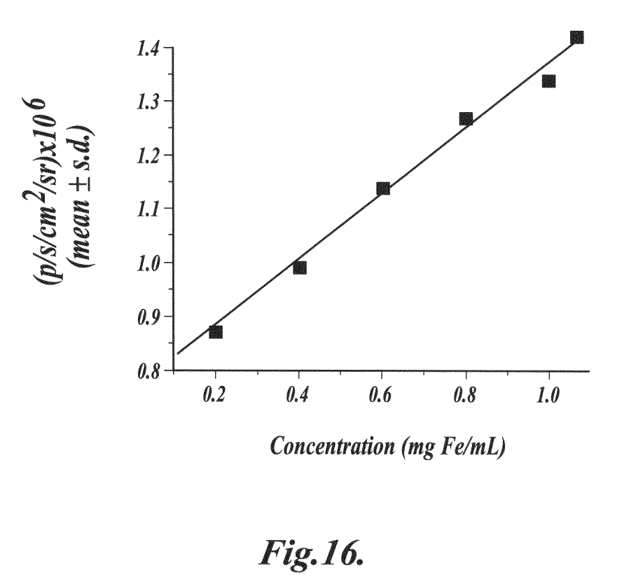

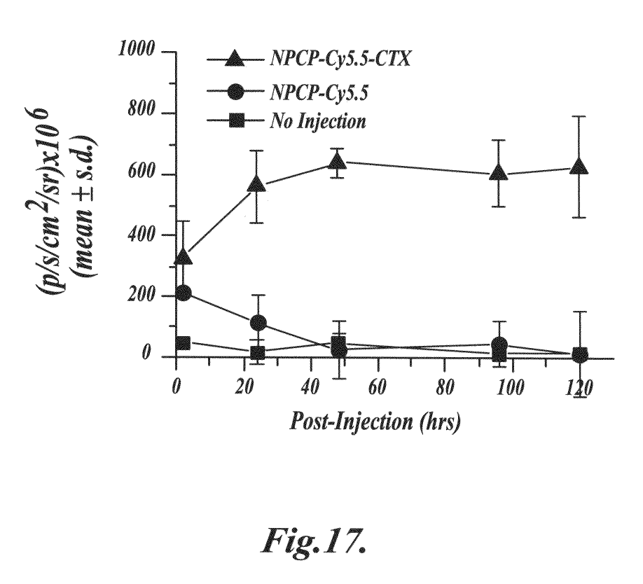

The potential of the NPCP-CTX nanoprobe to serve as an intraoperative optical contrast enhancement agent was assessed by both quantitative in vitro and in vivo imaging experiments using biophotonic fluorescence imaging. In vitro evaluation of the nanoprobe demonstrated a linear relationship between nanoprobe concentration and optical intensity (FIG. 16). In vivo, tumor labeling was assessed optically in symptomatic ND2:SmoA1 mice at 2 and 120 hrs post-injection with either NPCP-Cy5.5-CTX or its non-targeting variant, NPCP-Cy5.5, (FIGS. 8A and 8B, respectively). Preferential accumulation of the targeting nanoprobe was evident by the significant near infrared fluorescence (NIRF) signal, observed selectively in the tumor regions of the mice receiving NPCP-Cy5.5-CTX at 120 hrs post injection. Quantitative analysis of NIRF signal intensity revealed that accumulation of NPCP-Cy5.5-CTX nanoprobe in the brain tumor was complete 50 hrs after injection, and that no substantial signal intensity change was observed over the subsequent 70 hrs of analysis (FIG. 17). Low levels of NIRF signal in tumors of mice receiving NPCP-Cy5.5 were observed 2 hrs post injection, but there was no detectable signal at subsequent time points, indicating quick tissue clearance of the non-targeting nanoprobe. This experiment confirms the preferential accumulation of the targeting nanoprobe (NPCP-CTX) in tumors demonstrated in MRI imaging (FIG. 11A) and reveals the significant advantage of the targeting nanoprobe over non-targeting nanoprobe in probe retention in tumors.

Ex vivo images of NPCP-Cy5.5-CTX-treated mouse brains, excised immediately after the 120 hr time point, show a NIRF signal outlining the medulloblastoma tumor (FIG. 8B, inset) regions, demonstrating the selectivity and detectability of the probe necessary for potential intra-operative use during tumor resection. Concurrently, no significant levels of fluorescence were detected in the brains of non-injected control mice or those receiving the non-targeting probe, NPCP-Cy5.5. Combined with the substantially improved retention time of the NPCP-Cy5.5-CTX nanoprobe in the tumor tissue, these results illustrate the significance of utilizing targeting over non-targeting nanoprobes to provide localized contrast enhancement of the tumor. Furthermore, these results corroborate those obtained in the MRI experiment suggesting that NPCP-Cy5.5-CTX nanoprobes are able to penetrate the BBB and probe neural tissue for tumor cells.

The biodistribution of these nanoprobes in mice was determined by ex vivo NIRF signal quantification of excised tissues (FIG. 8C). In examining the tissue samples, there was no significant nanoprobe uptake of by healthy brain, heart, and muscle tissue (P>0.05) detected. However, similar levels of high nanoprobe accumulation was observed in the liver (P<0.0001), spleen (P<0.001), kidney (P<0.001) and lung tissues (P<0.0001) for both NPCP-Cy5.5-CTX and NPCP-Cy5.5 nanoprobes (P>0.05), comparable to relative values reported for other iron oxide nanoparticle systems. Within tumor tissue we observed significant accumulation of the NPCP-Cy5.5-CTX nanoprobe (P=0.015), but not the NPCP-Cy5.5 nanoprobe (P>0.05) demonstrating specificity of NPCP-Cy5.5-CTX for tumor tissue. Furthermore, the ability of the NPCP-Cy5.5-CTX nanoprobe to discriminate tumor from healthy tissue was evidenced by its preferential accumulation within tumor compared to normal brain tissue (P=0.0113).

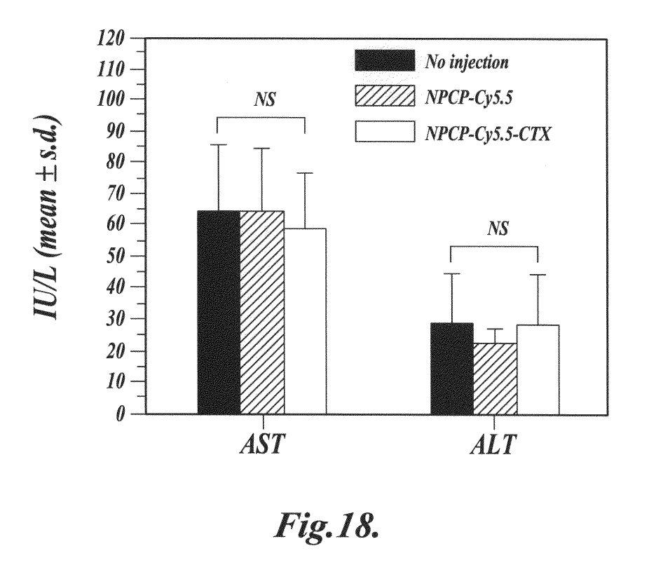

Toxicity assays performed confirmed that NPCP-Cy5.5-CTX has no toxic effect on liver tissue or the BBB, suggesting a safe cytotoxicity profile of this nanoprobe. Similarly, a radiopharmaceutical incorporating CTX was recently approved by the FDA for Phase I/II clinical trials as a human brain cancer therapy. Radiation dosimetry experiments in mice suggested an acceptable safety profile for this agent. Due to the relatively high-level of nanoprobe accumulation in the liver, a hepatotoxicity assay was performed. Serum levels of aspartate aminotransferase (AST) and alanine aminotransferase (ALT) of mice injected with NPCP-Cy5.5-CTX or NPCP-Cy5.5 nanoprobes were measured (FIG. 18). No significant elevation of AST (P>0.05) and ALT (P>0.05) levels was found in mice receiving either nanoprobe compared to non-injected controls suggesting that neither probe induces liver toxicity at the given dosage.

The ability of these nanoprobes to cross the BBB without compromising its integrity is unique for this iron oxide nanoparticle system. The BBB, which is comprised of brain capillaries lined with endothelial cells and intercellular tight junctions, has been shown to prevent 98% of all therapeutics from gaining access to the brain. Although the precise physical and chemical characteristics required to cross the BBB are still a subject of debate, our experience and data from the literature indicated that a physical size of <50 nm and a hydrodynamic size of <250 nm are required for the BBB passage. The nanoprobes of the invention fall within this requirement with a mean core size 7 nm as characterized by TEM (FIG. 5), and the mean hydrodynamic size of 33 nm as identified by DLS (FIG. 4). Combined with the surface properties attributed to our polymer these factors facilitate the ability of the nanoprobes to cross the BBB.

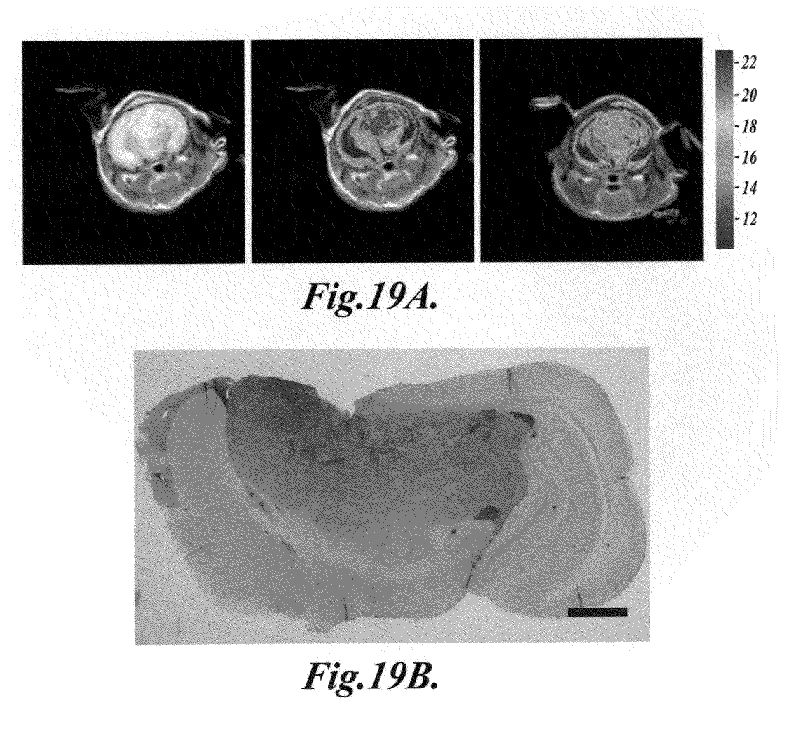

To confirm the apparent colocalization of the iron oxide nanoprobe with medulloblastoma tissue observed by MR imaging, an additional CTX-targeted tumor model was tested, the intracranial C6 glioma xenograft. The expedited growth of the xenograft model leads to larger consolidated tumor masses, readily distinguished by histology (FIG. 19B). MR imaging of these mice (FIG. 19A), post nanoprobe administration, showed defined contrast enhancement at the corresponding tumor site. The concentration of nanoprobes at the site of these large tissue targets demonstrates the ability of the nanoparticle system to identify a plurality of tumor types, as well as confirm selective localization within the brain.

The ability of the nanoprobes to remain localized specifically in brain tumors can be highly beneficial in clinical practice. Compared to current Gd-based MRI contrast agents that are rapidly cleared from the body, these nanoprobes have demonstrated persistent contrast enhancement for as long as 48 hrs by MRI (FIG. 11A) and up to 5 days by optical imaging studies (FIG. 8). Also notable in these optical imaging experiments is that the fluorescence signal was detected through the mice's intact skull and skin, highlighting the penetration depth of the NIRF signal (FIGS. 8A and 8B). This increased window of detection due to prolonged retention of the nanoprobe in targeted tumors, and the deep response of NIRF signals will allow for more versatile uses of these contrast agents, such as preoperative and postoperative diagnostics, tumor resection, as well as assessment of treatment response with either MR or optical imaging. With multi-modal detectability, surgeons could be able to correlate preoperative diagnostic images with intraoperative pathology.

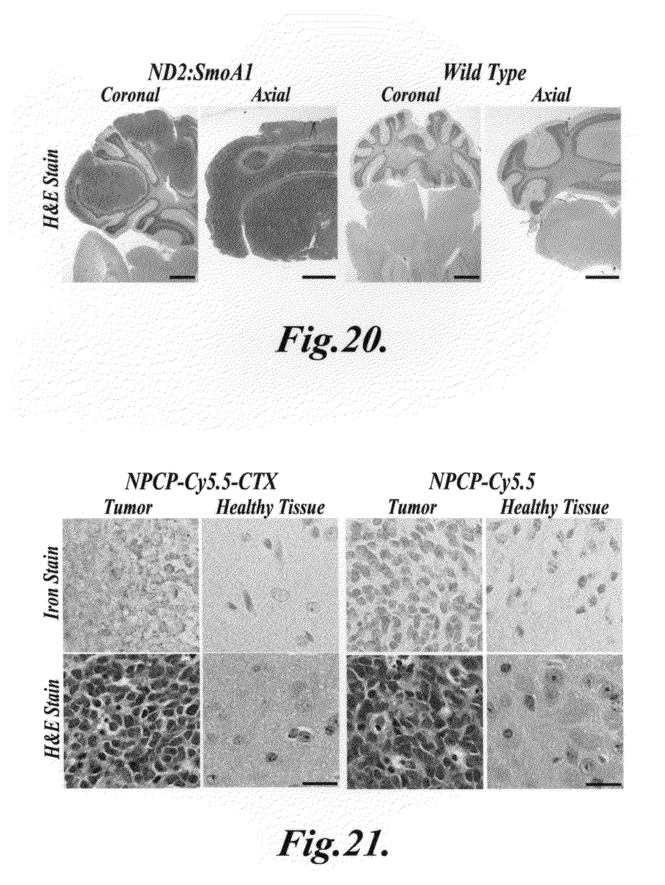

To verify the presence and evaluate the size of medulloblastoma tumors in the brains of symptomatic mice, histological samples were prepared from each mouse model studied. H&E stained coronal and axial cross sections of cerebellums from symptomatic ND2:SmoA1 and wild type mice are given in the left and right panels of FIG. 20, respectively. Medulloblastoma tissue was readily identified in samples from ND2:SmoA1 mice and absent in wild type mouse sections.

The specific accumulation of nanoprobes within tumor cells of ND2:SmoA1 mice was confirmed, histologically, through positive iron staining with Prussian blue. Specifically, ND2:SmoA1 mice were administered with NPCP-Cy5.5 or NPCP-Cy5.5-CTX via tail vein injection, sacrificed 120 hrs post injection, followed by brain removal, sectioning, and staining with either H&E or Prussian blue (FIG. 21). Tumor cells observed significant iron accumulation (blue) in mice receiving NPCP-Cy5.5-CTX, whereas healthy tissue showed no nanoprobe localization. The non-targeting, NPCP-Cy5.5, showed a minimal presence in the tumor region with no healthy tissue localization (FIG. 21). This selective tissue targeting of the CTX-enabled nanoprobe demonstrates its capability to navigate the brain vasculature and selectively bind tumor cells, and supports the observed MR and optical imaging enhancement by the targeting nanoprobe.

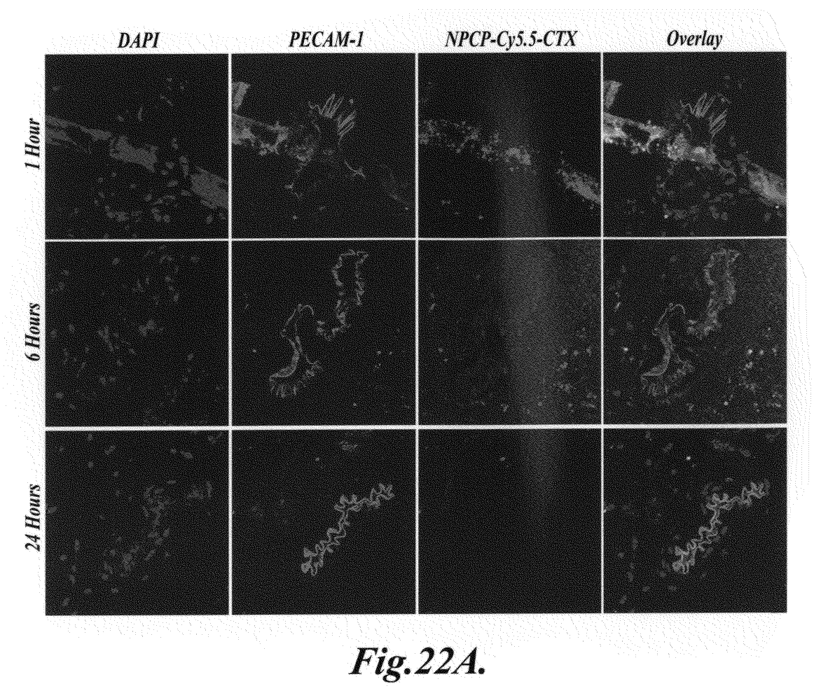

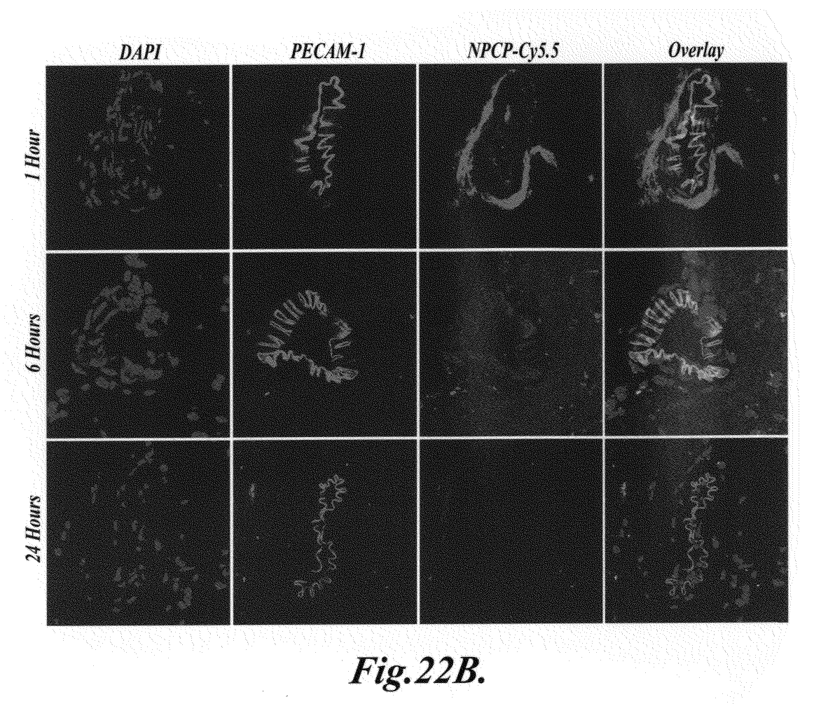

To further validate the ability of the native polymer-coated nanoparticle, and its targeting variant, to traverse the BBB, both nanoprobes were administered to wild type mice which bear no tumors and retain intact BBBs. Each mouse was injected with either NPCP-Cy5.5 or NPCP-Cy5.5-CTX via the tail vein. At 1, 6, and 24 hrs post injection mice were sacrificed, their brains removed and processed for histology. Tissue sections were stained with a fluorescently labeled antibody against platelet endothelial cell adhesion molecule-1 (PECAM-1; green), a glycoprotein expressed on endothelial cells, and with the nuclear stain, DAPI (blue), to elucidate the nanoprobe movement within the brain (FIGS. 22A and 22B). Both NPCP-Cy5.5-CTX and NPCP-Cy5.5 were detected in blood vessels of the brain 1 hr post injection, outside of the blood vessels after 6 hrs, and in limited quantities throughout the brain after 24 hrs. These observations indicate that both nanoprobes are capable of crossing an intact BBB.

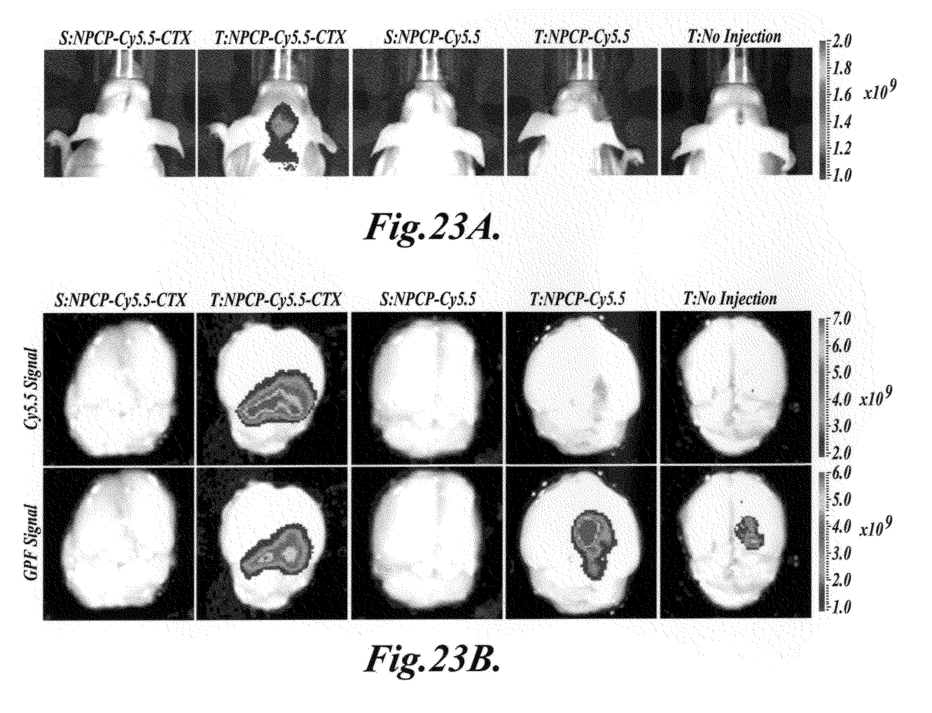

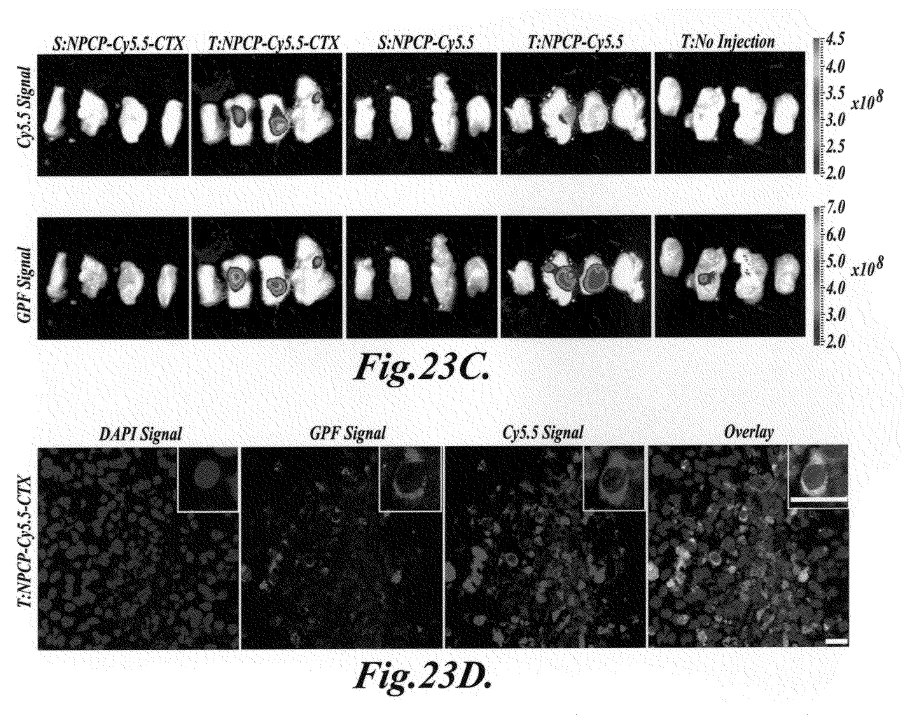

The applicability of the developed nanoprobe to target tumors of the neuroectodermal origin was further demonstrated, as an intracranial xenograft mouse model, using C6 glioma cells transfected to express green fluorescence protein (GFP), was evaluated. The C6 cell line is a classical model of the glial-derived tumor, representing the most common and lethal type of primary brain tumor. Identical procedures were used for nanoprobe administration for the C6 and ND2:SmoA1 models. The targeting nanoprobe (NPCP-Cy5.5-CTX) demonstrated tumor-specific accumulation by biophotonic imaging, and demonstrated improved tumor margin identification/discrimination in vivo (FIG. 23A), and ex vivo (FIGS. 23B and 23C). Selective nanoprobe binding to glioma cells was also shown by confocal microscopy of ex vivo brain sections (FIG. 23D). Additionally, MR imaging of the glioma model showed capability of the NPCP-Cy5.5-CTX to highlight tumor regions and boundaries in R2 maps (FIG. 19A).

The developed nanoprobe combines the capacities of specific targeting, BBB penetration, sustained retention in tumor cells, and localized contrast enhancement, while also retaining the flexibility to conjugate alternative targeting and therapeutic agents. This combination of biological and reporter capabilities represents a major advancement in tumor diagnostic and therapeutic technology and offer potential for clinical translation.

In summary, the present invention provides a targeting nanoprobe detectable by both magnetic resonance and biophotonic imaging. Through uptake assays, in vivo imaging, and histological and biodistribution analyses, the ability of the nanoprobe to cross the BBB and preferentially accumulate in the brain tumors of a genetically engineered and an intracranial xenograft C6 glioma mouse models has been demonstrated. The sustained retention in tumors and efficient clearance of the nanoprobe from normal brain tissue demonstrates its potential use for preoperative diagnosis and intraoperative tumor resection. With its innocuous toxicity profile and flexible conjugation chemistry for alternative diagnostic and therapeutic agents, this nanoprobe may represent a major advancement in brain tumor therapy.

The following examples are provided for the purpose of illustrating, not limiting, the invention.

### EXAMPLES

### Example 1

### The Preparation of Representative Nanoparticles

In this example, the preparations of representative nanoparticles of the invention are described: nanoparticles having polyethylene glycol-grafted chitosan nanoparticles (NPCP); fluorophore-labeled nanoparticles (NPCP-Cy5.5); and targeting agent-labeled nanoparticles (NPCP-Cy5.5-CTX). The preparations are illustrated schematically in FIGS. 2B, 3B, and 3C, respectively.

PEG-grafted chitosan (PEG-g-chitosan) synthesis. Chitosan used for this synthesis was obtained by oxidative degradation of as-received high molecular weight chitosan (Mw=190 kDa, Sigma, St. Louis, Mo.) with sodium nitrite (NaNO₂). The degradation was carried out by reacting 100 mM aqueous NaNO₂ solution with a 2 wt % chitosan solution (pH=4.5, dilute acetic acid) for 24 h at room temperature. PEG-g-chitosan (PEGylated chitosan) was prepared by alkylation of depolymerized chitosan followed by Schiff base formation. Methoxy PEG (Mn=2000 g/mole, Sigma Co.) was first oxidized into PEG-aldehyde and then reacted with primary amines of chitosan in the presence of sodium cyanoborohydride. The chemical structure and purity of the polymer were confirmed by HPLC and 1H-NMR.

Alternatively, oxidative degradation of chitosan was carried out by reacting aqueous/acidic chitosan solution with nitrous acid at room temperature. 1 to 3 wt % of commercially available chitosan (Mw=100 to 600 kDa) solution was prepared in an aqueous solution with a hydrogen ion concentration (pH) of 1 to 5. Aqueous nitrous acid solution (20 to 200 mM) was then added into the chitosan solution dropwise and the reaction mixture was stirred for 0.5 to 50 h. After completion of the reaction, solution was neutralized by addition of ammonia, sodium hydroxide, or an anion exchange resin. Lyophilized chitosan oligomer was washed with 50 to 90% ethanol and dried under vacuum.

Nanoparticle synthesis. NPCP were synthesized by first dissolving 3.0 g of PEG-g-chitosan, prepared as described above, in 50 ml deionized H₂O followed by addition of an iron chloride solution (4.6 g FeCl₂H₂O and 9.1 g FeCl₃ dissolved in 50 ml of deoxygenated deionized H₂O). This mixture was then heated to 40° C. under mechanical stirring and nitrogen bubbling. One hundred mL of 7% NH₄OH was then added to the polymer and iron chloride mixture at a rate of 100 ml per hr. The resulting black precipitate was dialyzed for 2-3 days in H₂O to remove unreacted reagents.

CTX and Cy5.5 conjugation. CTX (Alamone Labs, Jerusalem, Israel) and Cy5.5 (GE Healthcare, Piscataway, N.J.) were conjugated to the NPCP through the chemical scheme outlined in FIG. 3. Specifically, 1.75 mg of monoreactive Cy5.5 NHS ester was dissolved in 100 μL of anhydrous dimethyl formamide (DMF, Sigma, St. Louis, Mo.) and the solution was then added to 2 ml NPCP (2.5 mg of Fe/ml, suspended in 0.1 M sodium bicarbonate pH 8.5). The suspension was allowed to react for 2 hours prior to the addition of 100 μl of succinimidyl iodoacetate (SIA; Molecular Biosciences, Boulder, Colo.; 50 mg/ml, dissolved in anhydrous DMSO). The resulting solution was allowed to react for additional 2 hours. Excess Cy5.5 and SIA were removed from the suspension through gel chromatography using a Sephacryl S-200 column (GE Healthcare) equilibrated with 20 mM sodium citrate, and 0.15 M NaCl buffer at pH 8.0. CTX was functionalized with sulfhydryl groups through reaction with N-succinimidyl-S-acetylthioacetate (SATA, Molecular Bioscience, Boulder, Colo.). To perform this reaction 40 μl of SATA (1 mg/ml, dissolved in anhydrous DMSO) was added to a 1 ml solution of CTX (1 mg/ml, dissolved in 50 mM bicarbonate buffer, pH 8.5). After reaction for 1 hour at room temperature, excess SATA was removed by dialysis against PBS buffer (pH 7.4). Upon purification, SATA was deprotected by reacting 100 μl of a 25 mM hydroxylamine with 10 mM EDTA solution for 1 hour at room temperature. The resulting sulfhydryl modified peptide was then added to the Cy5.5 and SIA modified NPCP solution, and the mixture was allowed to react for 1 hour at room temperature. Unreacted CTX was removed from the suspension through gel filtration chromatography using Sephacryl S-200 column equilibrated with 20 mM sodium citrate, 0.15 M NaCl buffer at pH 8.0.

Characterization of nanoparticles. TEM grids were prepared by depositing a drop of the diluted nanoparticle suspension on 300 mesh silicon-monoxide support films and drying the grids under vacuum for 2 hours. TEM images were acquired on a Phillips 400 TEM operating at 100 KV. FTIR spectra were acquired using a Nicolet 5-DXB FTIR spectrometer with a resolution of 4 cm⁻¹. NPCP was lyophilized (Virtis Freezemobile), milled with KBr, and pressed into a pellet for analysis. Powder XRD patterns were acquired from lyophilized samples with a Philips 1820 X-ray diffractometer using Cu-Kα radiation (λ=1.541 Å) at 40 kV and 20 mA. Zeta potential and hydrodynamic size were measured in water using Zetasizer Nanoseries dynamic light scattering particle size analyzer (Malvern, Worcestershire, UK).

Magnetic properties studies of a representative nanoparticle. Phantom samples were prepared by mixing equal volumes of sample solutions (Feridex IV (Berlex), or NPCP) with low-melting 1% agarose gel (BioRad, Hercules, Calif.). Samples of each nanoparticle system were prepared over a range of concentrations (0.02, 0.2, 2, 20 and 200 μg Fe/mL). Sample suspensions were then loaded into a 12-well agarose sample holder and allowed to solidify at 4° C. Samples were sealed with additional agarose to avoid air susceptibility artifacts. MR imaging was performed on a 4.7-T Bruker magnet (Bruker Medical Systems, Karlsruhe, Germany) equipped with a Varian (Varian Inc., Palo Alto, Calif., USA) Inova spectrometer and a 5 cm home-built half volume RF coil in a loop gap resonator type. A conventional multi-spin echo pulse sequence was selected from the Varian VnmrJ software package. Repetition time (TR) of 3000 ms and variable echo times (TE) of 13.7, 16, 20, 40, 60, 90, 120 and 170 ms were used. The spatial resolution parameters were as follows: an acquisition matrix of 256×128, field of view of 40×40 mm, slice thickness of 1 mm, and 2 averages.

### Example 2

### Assessment of Cellular Uptake of Representative Nanoparticles in Vitro

Rat gliosarcoma (9L, ATCC, Manassas, Va.), rat glioma (C6, ATCC, Manassas, Va.), and mouse macrophage (RAW 264.7, ATCC, Manassas, Va.) cells were grown in DMEM supplemented with 1% streptomycin/penicillin and 10% fetal bovine serum (FBS). For NPCP uptake assays 3 million cells were seeded in 75 cm² flasks and incubated 48 hours. Cells were then washed with PBS and incubated in cell culture medium containing NPCPs. 9L cells were incubated with NPCP, NPCP-CTX, or dextran-coated nanoparticles for 2 hours at 37° C. at a concentration of 10 μg Fe/ml. For assessment of nanoparticle uptake by macrophage, RAW 264.7 cells were incubated for 48 hours. Cells were washed with PBS and incubated for 2 hours at 37° C. with nanoparticles coated with chitosan or PEG-g-chitosan at a concentration of 100 μg Fe/ml.

### Example 3

### Evaluation of Representative Nanoparticles in Vivo

Animal models. All mouse studies were conducted with procedures approved by the Institutional Animal Care and Use Committee at the University of Washington. Transgenic ND2:SmoA1 mice were generated on a C57BL/6 background (Charles River Laboratories Inc., Wilmington, Mass.) as described previously. Wild type mice refers to non genetically altered C57BL/6 mice. Intracranial xenografts were established in nu/nu mice (Charles River Laboratories Inc.) by stereotaxic injection of 1×10⁵ C6/GFP⁺ cells, suspended in 10 μL PBS, into the brain 3 mm lateral and 1 mm posterior to the bregma, at a depth of 3 mm from the dural surface. Cell injections and needle withdrawals were performed slowly (>20 min and 2 min, respectively). Mice were tested 14 to 17 days post-injection. Control mice received PBS injections, without cells, in the same manner described above.

In vivo MRI studies. Symptomatic mice were injected with nanoparticles at 10 mg/kg (n=3). Multi-echo multi-slice imaging was performed on a 4.7 T magnet (see above). Spin-spin relaxation time T2 maps were generated by multi-echo images with TE ranging from 14 to 68 ms.

In vivo optical imaging studies. Nanoparticles were administered intravenously by tail-vein injection to symptomatic mice (10 mg/kg) (n=3). Biophotonic fluorescence images were obtained on a Xenogen IVIS-100 system (Xenogen, Calif.). Mice were anesthetized with 2.5% isoflurane (VEDCO, Inc, MO) before they were placed in the imaging chamber and imaged at various time points post-injection. Relevant organs, tissues, and tumors were dissected from some of the animals and imaged immediately. All images were captured using identical system settings and fluorescence emission was normalized to photons per second per centimeter squared per steradian (p/s/cm²/sr).

Histology, immunofluorescence and DAPI staining. Whole brains of mice were removed immediately after the animals were sacrificed, embedded in Tissue-Tek OCT Compound (International Medical Equipment Inc., San Marcos, Calif.), and frozen on dry ice. The frozen brains were then cryostat sectioned along the axial plane into 20 μm sections, thawed, washed with PBS 3× to remove the OCT compound, and fixed in a 4% formaldehyde/PBS solution (methanol free; Polysciences Inc., Warrington, Pa.) for 30 min. Immunofluorescence staining sections were permeabilized with Triton X-100 (0.3% in PBS) for 1 hour, immersed in a blocking buffer (5% normal rat serum in PBS) for 1 hour, incubated in fluorescein isothiocyanate (FITC) antimouse CD31 (PECAM1) antibody (2 μg/ml in blocking buffer) solution overnight at 4° C., and washed with PBS thrice. DAPI staining/mounting sections were mounted with Prolong Gold Antifade solution containing DAPI (Invitrogen Inc., Carlsbad, Calif.) for cellular nuclei staining and fluorescence preservation.

MR image processing. The R2 maps for the in vivo mouse brain images were calculated from T2 maps, where R2 was taken as the inverse of T2, generated using a series of MR images acquired with various TE values at each imaging time point. NIH ImageJ was utilized to generate the T2 values based on the equation,

SI=A×e^{(-TE/T2)}+B,

where SI is the signal intensity, TE is the echo time, A is the amplitude and B is the offset. R2 changes were expressed as the difference between the R2 acquired at each imaging time point and the R2 acquired pre-injection.

Microscopy. Tissue sections stained with H&E or Prussian/nuclear fast red were micrographed using an E600 upright microscope (Nikon, Melville, N.Y.) equipped with a color CCD camera. Confocal fluorescence microscopy was performed with a Zeiss LSM 510 (Carl Zeiss Inc., Peabody, Mass.) microscope equipped with 405 nm Diode, 458 nm, 488 nm, 514 nm Argon/2, and 633 nm HeNe laser lines for excitation, and appropriate bandpass filters for collection of DAPI, FITC, and NIR emission signals. In all fluorescence images DAPI signal is depicted in blue, FITC or GFP signal in green, and NIR signal was translated into red pseudocolor to enable detection in the visible spectrum.

Statistical analysis. The data were expressed as mean, ±standard deviation of the mean. A paired t-test was used to determine the significance of nanoparticle accumulation as measured by MRI. Statistical significance of nanoparticle internalization in vitro cell assays was compared using a Student's t-test. Statistical significance in biodistribution and toxicity effects were determined using one-way analysis of variance (ANOVA) followed by a Student's t-test for multiple comparison tests. A P value <0.05 was considered as statistically significant.

Liver toxicity analysis. Serum aspartate aminotransferase (AST) and alanine aminotransferase (ALT) levels were quantified 120 hours after intravenous administration of NPCP-Cy5.5-CTX (n=3) or NPCP (n=3), and compared to symptomatic ND2:SmoA1 receiving no injection (n=3). 300 μl of blood was drawn from each mouse through retro-orbital bleeds. To obtain serum, the blood was allowed to coagulate for 45 min at room temperature and then serum was separated from the other blood components through centrifugation at 13,000 RMP for 20 min. AST and ALT levels in serum samples were quantified using commercially available diagnostic reagent sets (Pointe Scientific, Canton, Mich.) according to the manufacturers' instructions.

Assessment of the BBB integrity with Gd-DTPA. Spin-lattice relaxation time (T1) weighted MR imaging was performed for wild type and N2:SmoA1 mice before and after the injection of Gd-DTPA to confirm that the BBB remained intact in the tumor-bearing mouse model. T1-weighted images were consecutively acquired three times prior to Gd-DTPA injection to obtain baseline signal intensities. Serial acquisitions of T1-weighted images were conducted for approximately 75 min post intravenous injection of Gd-DTPA (0.1 mM/kg gadopentetate dimeglimine; 5× diluted Magnevist; Berlex Laboratories, Wayne, N.J.). The imaging parameters for T1-weighted images are as follows: TR (recycle delay)/TE (echo time)=500/12 ms/ms, number of averaging=1, matrix=256×128 and acquisition time=1 min. T2-weighted images were acquired prior to Gd-DTPA injection to visualize tumors in the cerebellum of the N2:SmoA1 mouse. T2-weighted images were acquired with the following parameters: TR/TE=3000/45 ms/ms, number of averaging=2, matrix=256×256. For pre- and post-injection imaging, a dual mouse holder was utilized to image two mice simultaneously.

Cell transfections. For transfection experiments, C6 cells were trypsinized, replated at 200,000 cells/mL in 6 well plates, and cultured for 18 hours prior to transfection. Cells were then transfected with p-EGFPN1 (Clontech Laboratories, Inc. Mountain View, Calif.) using Effectene Transfection Reagent (Qiagen Inc. Valencia, Calif.) according to the manufacturer's instructions. Following transfection, cells that stably expressed GFP were isolated through a two tiered process. First, 48 hours post-transfection, GFP⁺ cells were sorted using a BD FACS Aria cell sorter (BD Inc., Franklin Lakes, N.J.). Second, GFP⁺ cells isolated by sorting were further purified, chemically, by treating the cells for two weeks in media supplemented with G-418 Sulfate (1 mg/ml; A.G. Scientific Inc., San Diego, Calif.). The transfection efficiency of the population was determined to be greater than 90% using a BD FACS Canto flow cytometer (BD Inc.).

While illustrative embodiments have been illustrated and described, it will be appreciated that various changes can be made therein without departing from the spirit and scope of the invention.

The embodiments of the invention in which an exclusive property or privilege is claimed are defined as follows:

1. A nanoparticle, comprising:
(a) a core having a surface and comprising a core material; and
(b) a coating on the surface of the core, the coating comprising a copolymer comprising a chitosan and a poly(ethylene oxide) oligomer.

2. The nanoparticle of claim 1, wherein the core material is a magnetic material.

3. The nanoparticle of claim 1, wherein the core material is selected from the group consisting of ferrous oxide, ferric oxide, silicon oxide, polycrystalline silicon oxide, silicon nitride, aluminum oxide, germanium oxide, zinc selenide, tin dioxide, titanium, titanium dioxide, nickel titanium, indium tin oxide, gadolinium oxide, stainless steel, gold, and mixtures thereof.

4. The nanoparticle of claim 1, wherein the chitosan has an average molecular weight of from about 0.3 to about 50 kDa.

5. The nanoparticle of claim 1, wherein the poly(ethylene oxide) oligomer is selected from the group consisting of a poly(ethylene oxide) polymer and a poly(ethylene oxide) copolymer.

6. The nanoparticle of claim 1, wherein the poly(ethylene oxide) oligomer has an average molecular weight of from about 0.3 to about 40 kDa.

7. The nanoparticle of claim 1, wherein the copolymer comprises from about 2 to about 50 weight percent poly(ethylene oxide) oligomer.

8. The nanoparticle of claim 1, wherein the copolymer is a graft copolymer having a chitosan backbone and poly(ethylene oxide) oligomer side chains.

9. The nanoparticle of claim 8, wherein the graft copolymer has a degree of poly(ethylene oxide) oligomer substitution from about 0.01 to about 0.5.

10. The nanoparticle of claim 1 having a physical size less than about 50 nm.

11. The nanoparticle of claim 1 having a mean core size from about 2 to about 25 nm.

12. The nanoparticle of claim 1 having a hydrodynamic size less than about 250 nm.

13. The nanoparticle of claim 1 further comprising a targeting agent.

14. The nanoparticle of claim 13, wherein the targeting agent is selected from the group consisting of a small organic molecule, a peptide, a protein, and a nucleic acid.

15. The nanoparticle of claim 1 further comprising a fluorescent agent.

16. The nanoparticle of claim 15, wherein the fluorescent agent is a visible or near-infrared fluorescent agent.

17. The nanoparticle of claim 13 further comprising a fluorescent agent.

18. The nanoparticle of claim 1 further comprising a therapeutic agent.

19. The method of claim 18, wherein the therapeutic agent is a cytotoxic agent.

20. The nanoparticle of claim 13 further comprising a therapeutic agent.

21. The nanoparticle of claim 15 further comprising a therapeutic agent.

22. The nanoparticle of claim 17 further comprising a therapeutic agent.

23. A composition, comprising a nanoparticle of claim 1 and a carrier suitable for administration to a warm-blooded subject.

24. A method for detecting cells or tissues by magnetic resonance imaging, comprising:
(a) contacting cells or tissues of interest with a nanoparticle having affinity and specificity for the cells or tissues of interest, wherein the nanoparticle comprises
(i) a core comprising a magnetic material and having a surface,
(ii) a coating on the surface of the core, the coating comprising a copolymer comprising a chitosan and a poly(ethylene oxide) oligomer, and
(iii) a targeting agent covalently coupled to the copolymer, wherein the targeting agent has an affinity and specificity to the cells or tissues of interest; and
(b) measuring the level of binding of the nanoparticle, wherein an elevated level of binding, relative to normal cells or tissues, is indicative of binding to the cells or tissues of interest.

25. A method for treating a tissue, comprising contacting a tissue of interest with a nanoparticle having affinity and specificity for the tissue of interest, wherein the nanoparticle comprises
(i) a core comprising a core material and having a surface,
(ii) a coating on the surface of the core, the coating comprising a copolymer comprising a chitosan and a poly(ethylene oxide) oligomer, and
(iii) a targeting agent covalently coupled to the copolymer, wherein the targeting agent has an affinity and specificity to the cells or tissues of interest.
